(19) United States
(12) Patent Application Publication
(10) Pub. No.: US 2026/0314623 A1
(43) Pub. Date: Oct. 15, 2026

(54) BEVERAGE PRODUCTS INCLUDING BRAZZEIN SWEET PROTEIN

(71) Applicant: OOBLI, Inc., Davis, CA (US)

(72) Inventors: Jason Ryder, Oakland, CA (US); Allison Morgan Wing, Davis, CA (US); Kenneth Michael Amaral, Union City, CA (US); Gevenina Momoko Heimsoth, San Francisco, CA (US); Anthony Edward Salvucci, Davis, CA (US)

(21) Appl. No.: 19/484,208

(22) PCT Filed: May 17, 2024
(86) PCT No.: PCT/US2024/030038

§ 371 Date: Nov. 13, 2025

Related U.S. Application Data
(60) Provisional application No. 63/467,212, filed on May 17, 2023.
(60) Provisional application No. 63/613,990, filed on Dec. 22, 2023.

(51) Int. Cl. A23F 3/16; A23F 3/40; C07K 14/415; C12N 1/165; C12R 1/84

(52) U.S. Cl. CPC A23F 3/16; A23F 3/405; C07K 14/415; C12N 1/165; A23F 2200/00; C12R 2001/84

(57) ABSTRACT

Described herein are beverage product formulations, including teas and concentrates thereof, that include at least one recombinant sweet protein, and methods of producing the same.

### CLAIM OF PRIORITY

This application claims the benefit of U.S. Provisional Application Ser. No. 63/467,212, filed on May 17, 2023, and U.S. Provisional Application Ser. No. 63/613,990, filed on Dec. 22, 2023. The entire contents of the foregoing are hereby incorporated by reference.

### REFERENCE TO SEQUENCE LISTING SUBMITTED ELECTRONICALLY

This application contains a Sequence Listing that has been submitted electronically as an XML file named 54282-0010WO1_ST26_SL.XML.” The XML file, created on Apr. 26, 2024, is 5,622 bytes in size. The material in the XML file is hereby incorporated by reference in its entirety.

### TECHNICAL FIELD

This invention relates to compositions including sweet proteins. In particular, this invention relates to food formulations, compositions, and products including sweet proteins (e.g., brazzein and monellin).

### BACKGROUND

Various sweeteners are available, including naturally-occurring carbohydrates, such as those obtainable from plants, and artificial sweeteners. However, high levels of sugar consumption have been linked to obesity and associated health problems, including an increased risk of high blood pressure, high cholesterol, Type 2 diabetes, and cardiovascular disease. Yet, sweet food products are favored by a large proportion of the population. Therefore, there is a need to provide healthy sweetener alternatives that deliver similar or improved qualities to currently available sugar and sugar alternatives, such as artificial sweeteners.

### SUMMARY

There is a need for alternative sweeteners and sweetened formulations that are healthier alternatives to available sweeteners such as sugar and sugar alternatives, particularly to provide sweeteners that don't affect blood sugar levels and/or modulate the gut microbiome. In addition, there is a need to deliver not only an acceptable level of sweetness, but also the timing and longevity of sweetness that make sweet foods desirable, and even craveable.

Provided herein are tea products and formulations thereof with at least one sweet protein, such as brazzein-53, brazzein-54, or monellin, including a tea beverage including a tea, a plant-based sugar composition, and a recombinant brazzein. In some cases, the recombinant brazzein is present in an amount between 8 ppm and 40 ppm, and the plant-based sugar composition is present in an amount between 1-3% (vol/total vol). In some cases, the recombinant brazzein is present in an amount between 10 ppm and 30 ppm. In some cases, the recombinant brazzein is recombinant brazzein-53. In some cases, the recombinant brazzein is produced by a yeast or filamentous fungus.

In some cases, the plant-based sugar composition comprises agave syrup. In some cases, the plant-based sugar composition comprises fructose and glucose. In some cases, the tea beverage does not include any high intensity sweeteners selected from the group consisting of rebaudioside A, rebaudioside B, rebaudioside C, rebaudioside D, rebaudioside E, rebaudioside F, rebaudioside I; Rebaudioside J, rebaudioside K, rebaudioside M, rebaudioside N, rebaudioside O, rebaudioside Q, rebaudioside R, dulcoside A, dulcoside C, rubusoside, steviol monoside, steviol bioside, stevioside, mogroside V, allulose, aspartame, sugar alcohols, and saccharin. In some cases, the composition does not comprise cane sugar.

In some cases, any of the tea beverages described herein also include an acid component. In some cases, the acid component is selected from malic acid, citric acid, and lemon juice.

In some cases, any of the tea beverages described herein also include a flavoring or flavoring agent.

In some cases, any of the tea beverages described herein also include a fruit puree.

In some cases, the tea is a black tea, a green tea, a white tea, or any combination thereof. In some cases, the BRIX is between 2° Bx and 3° Bx. In some cases, the pH is between 3 and 5, between 3 and 4, or between 3.3 and 3.6. In some cases, the specific gravity is between 1.000 and 1.020 or between about 1.010 and 1.015.

Also provided herein are tea beverages including a tea, a plant-based sugar composition and at least one recombinant sweet protein, where the recombinant sweet protein is present in an amount between 1 ppm and 175 ppm, and where the plant-based sugar composition is present in an amount between 1-3% (vol/total vol).

In some cases, the at least one recombinant sweet protein comprises brazzein-53. In some cases, the brazzein-53 is present in an amount between 1 ppm and 96 ppm. In some cases, the brazzein-53 is present in an amount between 5 ppm and 48 ppm. In some cases, the brazzein-53 is the only sweet protein in the tea beverage. In some cases, the brazzein-53 is present in an amount between 8 ppm and 96 ppm or between 16 ppm and 48 ppm.

In some cases, any of the tea beverages described herein also include recombinant brazzein-54. In some cases, the brazzein-54 is present in an amount between 2 ppm and 62 ppm. In some cases, the brazzein-54 is present in an amount between 8 ppm and 36 ppm.

In some cases, any of the tea beverages described herein also include recombinant monellin. In some cases, the monellin is present in an amount between 2 ppm and 76 ppm. In some cases, the monellin is present in an amount between 9 ppm and 43 ppm.

In some cases, at least one recombinant sweet protein includes brazzein-54. In some cases, the brazzein-54 is present in an amount between 2 ppm and 142 ppm. In some cases, the brazzein-54 is present in an amount between 8 ppm and 71 ppm. In some cases, the brazzein-54 is the only sweet protein in the tea beverage. In some cases, the brazzein-54 is present in an amount between 12 ppm and 142 ppm or between 24 ppm and 71 ppm.

In some cases, any of the tea beverages described herein also includes recombinant monellin. In some cases, the monellin is present in an amount between 2 ppm and 76 ppm. In some cases, the monellin is present in an amount between 9 ppm and 43 ppm.

In some cases, the at least one recombinant sweet protein includes monellin. In some cases, the monellin is present is an amount between 2 ppm and 173 ppm. In some cases, the monellin is present is an amount between 9 ppm and 86 ppm.

In some cases, the monellin is the only sweet protein in the tea beverage. In some cases, the monellin is present is an amount between 15 ppm and 173 ppm. In some cases, the monellin is present is an amount between 29 ppm and 86 ppm.

In some cases, the tea is a black tea, a green tea, a white tea, a fruit-flavored or any combination thereof. In some cases, the tea beverage is a tea-lemonade and includes the at least one sweet protein is recombinant brazzein-53 in an amount between 4 ppm and 96 ppm; or the at least one sweet protein is recombinant brazzein-54 in an amount between 6 ppm and 142 ppm; or the at least one sweet protein is recombinant monellin in an amount between 7 ppm and 173 ppm.

In some cases, the tea beverage is a tea-lemonade and includes the at least one sweet protein including a single sweet protein, where the single sweet protein is recombinant brazzein-53 in an amount between 24 ppm and 96 ppm; or the single sweet protein is recombinant brazzein-54 in an amount between 35 ppm and 142 ppm, or the single sweet protein is recombinant monellin in an amount between 43 ppm and 173 ppm.

In some cases, any of the tea beverages described herein also includes a flavoring or a flavoring agent.

In some cases, any of the tea beverages described herein also includes a fruit puree.

In some cases, the at least one recombinant sweet protein is produced by a microbial organism. In some cases, the microbial organism is a yeast or a filamentous fungus. In some cases, the microbial organism is a Pichia sp.

In some cases, any of the tea beverages described herein includes a plant-based sugar composition. In some cases, the plant-based sugar composition includes agave syrup. In some cases, the plant-based sugar composition comprises fructose and glucose.

In some cases, the tea beverage includes a high intensity sweetener. In some cases, the tea beverage does not include any high intensity sweeteners selected from the group consisting of rebaudioside A, rebaudioside B, rebaudioside C, rebaudioside D, rebaudioside E, rebaudioside F, rebaudioside I; Rebaudioside J, rebaudioside K, rebaudioside M, rebaudioside N, rebaudioside O, rebaudioside Q, rebaudioside R, dulcoside A, dulcoside C; rubusoside, steviol monoside, steviol bioside, stevioside, mogroside V, allulose, aspartame, sugar alcohols, and saccharin.

In some cases, any of the tea beverages described herein includes an acid component. In some cases, the acid component is selected from malic acid, citric acid, and lemon juice.

In some cases, the BRIX of any of the tea beverages described herein is between 2° Bx and 4.3° Bx, between 2° Bx and 3° Bx, or between 3.9° Bx and 4.3° Bx. In some cases, the pH of any of the tea beverages described herein is between 3 and 5, between 3 and 4, or between 3.3 and 3.6. In some cases, the specific gravity of any of the tea beverages described herein is between 1.000 and 1.020 or between about 1.010 and 1.015.

In some cases, any of the tea beverages described herein does not include cane sugar.

Also provided herein are tea concentrates, where when diluted with water results in a tea beverage as set forth above.

In some cases, the tea concentrates are between IX and 10× concentrated as compared to the tea beverage.

The details of one or more cases of the invention are set forth in the accompanying drawings and the description below. Other features, objects, and advantages of the invention will be apparent from the description and drawings, and from the claims. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DETAILED DESCRIPTION

Described herein are beverage products, in particular tea beverages, that include a sweet protein, such as brazzein-53, brazzein-54, monellin or any combinations thereof. Also described are concentrates of tea beverages that can be diluted with water to create tea beverages.

### I. Taste and Taste Receptors

Taste is a form of chemoreception which occurs in taste receptors in the mouth and is one of a variety of mechanisms used by mammals to sense stimuli and the external environment. Humans and other mammals have taste receptors on taste buds of the tongue and other areas, including the epiglottis. Each taste bud can have a pore that opens out to the surface of the tongue, enabling molecules and ions taken into the mouth to reach the receptor cells inside.

The taste sensations, which include salt, sour, sweet, bitter, and umami, can be sensed by one or more mechanism(s) and by one or more taste receptor(s). Taste receptors involved in the sensation of taste include Type 1 taste receptors (TAS1, e.g., TAS1R2 and TAS1R3) and Type 2 taste receptors (TAS2, e.g., TAS2R1-TAS2R50). Type 1 taste receptors are generally associated with sweetness and Type 2 taste receptors are generally associated with bitterness.

A taste can change over the process of consuming a beverage product. A tasting profile can be described as having a starting taste/flavor, for example an initial sensation of sweetness, a curve or shape (such as a Gaussian curve or roundness) of sweetness from start to finish (or a portion thereof) and a finishing taste/flavor. A starting taste and finishing taste can have a temporal element, i.e., how fast the starting taste, such as sweetness, is detected by a subject or how long the finishing flavor is detectable by a subject. A finishing taste/flavor can be described as lingering, meaning the finishing taste/flavor such as sweetness, is discernable by a subject for a long time (e.g., 5 second, 10 seconds, 30 seconds, or 1 minute) after swallowing the beverage product responsible for causing the taste. A curve or shape of the sweetness provides a profile of the timing and magnitude of sweetness of the course when the sweetness in discernible. The presence, amounts, and ratios of one or more sweet proteins in a tea beverage product can affect the taste profile (e.g., sweetness characteristics) of the tea beverage product.

### II. Sweet Proteins

Sweet proteins (also referred to herein as sweet polypeptides), such as brazzein-53, brazzein-54 and monellin, can be formulated into a variety of products to achieve a desired level and timing of sweetness. In some cases, the sweet polypeptide can yield a sweet taste when consumed by a subject (e.g., a human). The present disclosure describes compositions formulations, and products with a sweet polypeptide, such as brazzein-53 brazzein-54, monellin, each alone or in various combinations. In some implementations of the formulations described herein, the sweet polypeptide is a recombinant sweet protein that, for example, can impart, elicit, and/or enhance a sweet taste when formulated into products such as the tea beverages described herein.

In some cases, the present disclosure describes the sweet protein, such as brazzein-53, brazzein-54 and monellin, that can generate a sweet taste and/or enhance sweetness when tasted or consumed by humans. In some cases, the sweet protein can be sweet-tasting and can taste sweet, for example, to a human subject. The disclosed sweet protein can generate a sweet taste and/or enhance a sweet taste of a beverage product, for example, as an ingredient or component of a composition, formulation, or product.

A sweet polypeptide described herein can have a sweeter taste than naturally occurring sugars, sugar alternatives and/or artificial sweeteners. In some cases, the sweetness of the brazzein-53, as determined by a human subject, for example, in a side-by-side gustatory comparison, can be greater than that of sugars, sugar alternatives and/or artificial sweeteners. In some embodiments, a sweet polypeptide described herein can have an equivalent or greater level of sweetness than a naturally occurring sugar, a sugar alternative and/or an artificial sweetener, and additionally do so without affecting glycemic index (e.g., blood sugar levels) and without modifying the gut microbiome.

The terms “polypeptide,” “peptide,” and “protein” are used interchangeably herein to refer to polymers of amino acids of any length. The polymer can be linear or branched, it can include modified amino acids (e.g., pyroglutamate (pGlu)), and/or it can be interrupted by non-amino acids. The terms also encompass an amino acid polymer that has been modified, for example, disulfide bond formation, glycosylation, lipidation, acetylation, phosphorylation, or any other manipulation, such as conjugation with a labeling component. As used herein, the term “amino acid” refers to cither natural and/or unnatural or synthetic amino acids, including pyroglutamate (pGlu) and both the D or L optical isomers, amino acid analogs, and/or peptidomimetics.

### Organoleptic Properties of Sweet Proteins

The amount of sweet protein (brazzein-53, brazzein-54, monellin or combinations thereof) present in any of the tea beverage products disclosed herein is any amount that confers one or more improved organoleptic properties of the beverage product to which the sweet protein combination is added (e.g., taste, sight, smell, and texture (e.g., mouthfeel) and/or health-related properties (e.g., less change or no change in glycemic index, or less modification or no modification of the gut microbiome, as compared to a similar tea beverage made with sugar or other artificial sweeteners). In a particular case, the improved organoleptic property is associated with taste. In one case, improving one or more organoleptic property results in the improvement of the taste profile. The overall taste profile of a beverage product is an interplay of several different tastes and senses, such as sweetness, sourness, bitterness, and the like. Examples of improved organoleptic properties can include, for example, a reduction in bitterness, a reduction in astringent and/or licorice notes, faster onset of sweetness, slower onset of sweetness, a reduction in lingering sweetness, a reduction in lingering bitterness, a reduction in bitter aftertaste, a reduction in metallic aftertaste, a reduction in chemical and synthetic aftertaste, a roundness to the sweetness profile, and a combination thereof.

In a particular case, the term “improved organoleptic properties” means that the tea beverage product will have one or more improved organoleptic properties for the majority of users, i.e., between about 50% to about 100% of a group of humans (e.g., between about 60% to about 100%, between about 70% to about 100%, between about 80% to about 100%, or between about 90% to about 100% of a group of humans). The improvement can be expressed qualitatively, e.g., as a gustatory comparison or a comparison taste test, or quantitatively, e.g., as a percentage improvement or as a value on a comparative scale. For example, a human or a group of humans can compare any of the tea beverage products described herein to a similar commercially available product, to any of the other tea beverage products described herein, or to a similar beverage product made with sugar (e.g., cane sugar). Any of the tea beverage products described herein can have any of the improved organoleptic properties, as determined by any of the methods described herein. For example, any of the tea beverage products described herein can have an increase of sweetness, a reduction of sweetness, a reduction in bitterness, a faster onset of sweetness, or a reduction in lingering sweetness.

In some embodiments, the amount of sweet protein present in any of the tea beverage products disclosed herein is any amount that provides a desired level, curve and/or timing of sweetness and additionally provides a health-related property such as less change or no change in glycemic index and/or less modification or no modification of the gut microbiome, as compared to a tea beverage product made with sugar or other artificial sweeteners.

### Types of Sweet Proteins

### Brazzein and Recombinant Brazzein

In some cases, the sweet protein is brazzein-53. In some cases, the sweet protein is brazzein-54. Brazzein is a small, sweet-tasting protein originally isolated from the fruit of West African plant known as the Oubli plant (Pentadiplandra brazzeana Baillon). (See, for example, Ming D et al., FEES Left. (1994) 20 355:106-108, incorporated by reference in its entirety). It is a monomer protein with a molecular weight of 6.5 kDa. As a member of the CsBα fold family, it contains four disulfide bonds that lend a high degree of thermal and pH stability to its structure. Specifically, the sweet taste of brazzein remains after incubation at 98° C. for 2 hours and at 80° C. for 4.5 hours in the pH range of 2.5-8. It is also water soluble (>50 mg/mL). It is a highly soluble protein (more than 50 g/L) with an isoelectric point of 5.4. Residues 29-33, 36, and 39-43, as well as the C-terminus of the protein, can be involved in the sweet taste of the protein. The charge of the protein can also play a role in its interaction with the sweet taste receptor.

At least three forms of the protein are known, although only two forms are present in ripe fruit. The major form is 54 amino acids long (pGlu-brazzein, referred to as “brazzein-54”), is about 80% of the total brazzein protein content in ripe fruit, and contains a pyroglutamate (pGlu) residue at its N-terminus (SEQ ID NO: 2, Table 1). The minor form is 53 amino acids long (des-pGlu-brazzein), referred to as “brazzein-53” herein), is about 20% of the total brazzein protein in ripe fruit, and lacks the final pyroglutamate residue (SEQ ID NO: 1, Table 1).

On a weight basis, brazzein can taste 500 to 2000 times sweeter than sucrose, as compared to a 10% sucrose and a 2% sucrose solution, respectively, or for example about 700 times sweeter when compared to a 5% sucrose solution. Sucrose, a commonly used sweetener, is a disaccharide molecule composed of glucose and fructose.

As described below, brazzein in the tea beverage can be the minor form (i.e., brazzein-53) or the major form (i.e., brazzein-54). In addition, brazzein can be naturally produced by the fruit of the Oubli plant or produced recombinantly by, for example, microbial fermentation of a bacterium, a yeast, or a filamentous fungus that is genetically engineered to produce recombinant brazzein-53 or recombinant brazzein-54.

### Monellin and Recombinant Monellin

In some cases, the sweet protein used herein is a monellin. Monellin refers to a sweet protein originally discovered in the fruit of the West African shrub known as serendipity berry (Dioscoreophyllum cumminsii). Monellin consists of two non-covalently associated polypeptide chains, an A chain of 44 amino acid residues (SEQ ID NO: 3, Table 1) and a B chain of 50 amino acid residues (SEQ ID NO: 4, Table 1). As used herein, “monellin” is a fusion of chain A and chain B with a single glycine as a linker, referred to as a single-chain monellin (SEQ ID NO:5, Table 1). Monellin has a secondary structure of five beta-strands that forms an antiparallel beta-sheet and a 17-residue alpha-helix. On a molar basis, monellin may taste up to 100,000 times sweeter than commonly used sugars. On a weight basis, monellin may taste several thousand times sweeter that commonly used sugars. For example, monellin may taste between 800-2000 times than a 5-7% (w/w) sucrose solution.

As described below, monellin in the tea beverage can be monellin or single-chain monellin. In addition, monellin can be naturally produced by the fruit or produced recombinantly by, for example, microbial fermentation of a bacterium, a yeast, or a filamentous fungus that is genetically engineered to produce recombinant monellin.

TABLE 1
Exemplary Sweet Polypeptide Sequences

| SEQ ID | Peptide | |
|---|---|---|
| NO: | Description | Sequence |
| 1 | Brazzein-53 | DKCKKVYENYPVSKCQLANQCNYDCKLDKHARSGECFY |
| | | DEKRNLQCICDYCEY |
| 2 | Brazzein-54 | XDKCKKVYENYPVSKCQLANQCNYDCKLDKHARSGECF |
| | | YDEKRNLQCICDYCEY |
| 3 | Monellin | FREIKGYEYQLYVYASDKLFRADISEDYKTRGRKLLRFNG |
| | chain A | PVPPP |
| 4 | Monellin | GEWEIIDIGPFTQNLGKFAVDEENKIGQYGRLTFNKVIRPC |
| | chain B | MKKTIYEEN |
| 5 | Single-chain | GEWEIIDIGPFTQNLGKFAVDEENKIGQYGRLTFNKVIRPC |
| | monellin | MKKTIYEENGFREIKGYEYQLYVYASDKLFRADISEDYKT |
| | | RGRKLLRFNGPVPPP |

X can be glutamine or pyroglutamate

### III. Methods of Producing Sweet Protein

### Recombinant Sweet Protein Expression

Sweet proteins, such as brazzein-53, brazzein-54 and monellin, described herein can be isolated from naturally occurring sources and/or produced recombinantly by a variety of protein expression systems, including, but not limited, cell-based expression systems, and cell-free expression systems. Sweet proteins produced recombinantly are recombinant proteins.

As used herein “recombinant protein” is a protein produced from a recombinant nucleic acid. A “recombinant nucleic acid” is a sequence that is not naturally occurring or has a sequence that is made by an artificial combination of two or more otherwise separated segments of nucleic acid sequence. This artificial combination is often accomplished by chemical synthesis or by the artificial manipulation of isolated segments of nucleic acids (e.g., by genetic engineering techniques). The term recombinant nucleic acids includes nucleic acids that have been altered solely by addition, substitution, or deletion of a portion of the nucleic acid. Frequently, a recombinant nucleic acid can include a nucleic acid sequence operably linked to a promoter sequence. Such a recombinant nucleic acid can be part of a vector that is used, for example, to transform a cell.

Non-limiting examples of protein expression systems useful in producing sweet polypeptides disclosed herein (e.g., brazzein-53, brazzein-54, and/or monellin (such as a single chain monellin)) include prokaryotic cell-based expression systems (e.g., archaeal systems, bacterial systems) and eukaryotic cell-based expression systems (e.g., filamentous or non-filamentous fungal cells, yeast cells, insect cells, and mammalian cells). In some cases, recombinant sweet protein is produced by a filamentous fungus. In some cases, recombinant sweet protein is produced by a yeast. In some cases, recombinant sweet protein is produced by a Pichia species, such as Pichia pastoris (also known as Komagataella phaffii).

To express any of the sweet polypeptides disclosed herein in a cell-based expression system, a gene encoding for the polypeptide can be introduced into a host cell of the expression system by, for example, an expression vector. A polynucleotide gene encoding for the sweet protein can be made by various methods, including molecular cloning and synthesis. Molecular cloning methods can involve mutagenesis (e.g., site-directed mutagenesis), restriction enzyme-mediated cloning (e.g., restriction enzyme digestion and ligation), polymerase chain reaction (PCR), and overlap extension. Synthesis can include chemical synthesis (e.g., gone synthesis). The gene sequence can be codon optimized for any desired expression system.

In some cases, a vector including a nucleic acid sequence encoding a sweet polypeptide disclosed herein is provided. In some cases, a vector including a nucleic acid sequence encoding for the sweet polypeptide brazzein (e.g., a nucleic acid encoding a recombinant brazzein-53, a recombinant brazzein-54) and/or a monellin protein (e.g., a nucleic acid encoding a recombinant monellin (such as a single chain monellin)) is provided.

The coding sequence can be inserted into a vector by a variety of procedures, including, but not limited to restriction enzyme digestion, ligation, and homologous recombination. The vector can be capable of replicating and expressing the polynucleotides in prokaryotic and/or eukaryotic host cells of expression systems. A vector can contain various components that can be adjusted and optimized for compatibility with the particular host cell. A cloning vector and/or expression vector can include additional nucleic acid sequences, including but not limited to, a signal sequence, an origin of replication, a marker gene (e.g., a selection marker such as an antibiotic resistance gene), an enhancer element, a promoter, a ribosome binding site, a signal sequence, and a transcription termination sequence.

In some cases, an expression vector containing a polynucleotide encoding a sweet protein, such as brazzein-53, brazzein-54, and/or monellin as disclosed herein, can further include a promoter. Promoters include, but are not limited to, constitutive promoters, inducible promoters, and hybrid promoters. Promoters can include, but are not limited to, acu-5, adhl+, alcohol dehydrogenase (ADH1, ADH2, ADH4), AHSB4m, AINV, alcA, α-amylase, alternative oxidase (AOD), alcohol oxidase 1 (AOX1), alcohol oxidase 2 (AOX2), AXDH, B2, CaMV, cellobiohydrolase I (cbhl), ceg-1, cDNAI, cellular filament polypeptide (cfp), cpe-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, invit, isocitrate lyase (ICL1), acetohydroxy acid isomicroreductase (ILV5), KAR2, KEX2, b-galactosidase (lac4), LEET2, melO, MET3, methanol oxidase (MOX), nmtl, NSP, pcbC, PET9, peroxin 8 (PEX8), phosphoglycerate kinase (PGK, PGK1), phol, PH05, PH089, phosphatidylinositol synthase (PIS1), PYK1, pyruvate kinase (pki1). RPS7, sorbitol dehydrogenase (SDH), 3-phospho serine aminotransferase (SERI), SSA4, SV40, TEF, translation elongation factor 1 alpha (TEF1), THI11, homoserine kinase (THR1), tpi. TPS1, triose phosphate isomerase (TPI1), XRP2, and YPT1.

In some instances, an expression vector containing a gene encoding a sweet polypeptide or protein disclosed herein (e.g., brazzein-53, brazzein-54, and/or monellin) can include a polynucleotide sequence encoding a signal peptide. A signal peptide, also known as a signal sequence, targeting signal, localization signal, localization sequence, secretion signal, transit peptide, leader sequence, or leader peptide, can support secretion of a protein or polynucleotide. Extracellular secretion of a recombinantly expressed protein from a host cell can facilitate protein purification. For example, recovery of a recombinant protein from a cell culture supernatant can be preferable to lysing host cells to release a complex mixture of proteins, including intracellular proteins of the host cell. Secretion, in some cases, can reduce deleterious effects that intracellular overexpression of a heterologous protein can have on a host cell such as toxicity or decreased growth rate. Secretion, in some cases, can allow increased protein production compared to intracellular expression in a host cell of limited volume to store the synthesized proteins. Secretory production of a protein, in some cases, can facilitate post-translational modification or processing (e.g., protein folding, formation of disulfide bonds, and glycosylation).

As different host cells can have characteristics and specific mechanisms for the posttranslational processing and modification of protein products, appropriate cell lines or host systems can be chosen to ensure the desired modification and processing of the expressed protein. In some cases, a host cell can be selected from the group consisting of: bacteria, fungi, plant cells, insect cells, mammalian cells, and any combination thereof. A host cell can be grown, fermented, or cultured in any appropriate medium that allows for host cell growth and sweet protein (e.g., recombinant brazzein-53, a recombinant brazzein-54 or a recombinant monellin (such as a single chain monellin)) expression.

In some cases, a host cell selected for expression of the sweet polypeptide (e.g., brazzein-53) disclosed herein is an archaeal cell. Non-limiting examples of archaeal host cells include Pyrococcus furiosus, Metallosphera sedula, Thermococcus litoralis, Methanobacterium thermoautotrophicum, Methanococcus jannaschii, Pyrococcus abyssi, Sulfolobus solfataricus, Pyrococcus woesei, Sulfolobus shibatae, and variants thereof.

In some cases, a host cell selected for expression a sweet polypeptide (e.g., brazzein-53, brazzein-54, and/or monellin) disclosed herein is a bacterial cell. Suitable bacterial bost cells include, but are not limited to, BL21 E. coli, DE3 strain E. coli, E. coli M15, DH5u, DH103, HB101, B. subtilis cells, Pseudomonas fluorescens cells, and cyanobacterial cells such as Synechococcus elongates cells.

In some cases, a host cell selected for expression of a sweet polypeptide (e.g., brazzein-53, brazzein-54, and/or monellin) disclosed herein can be a fungal cell. The fungal cell can be a yeast cell or a filamentous fungi cell. A yeast or filamentous fungal cell can include, but is not limited to, Arxula spp., Arxula adeninivorans, Kluyveromyces spp., Kluyveromyces lactis, Pichia spp., Pichia angusta, Pichia pastoris (also known as Komagataella phaffii), Saccharomyces spp., Saccharomyces cerevisiae, Schizosaccharomyces spp., Schizosaccharomyces pombe, Tetrahymena sp., Hansemila sp., Blastobotrys sp., Candida sp., Zygosaccharomyces sp., Debaryomyces sp. Yarrowia spp., and Yarrowia lipolytica. Fungi can include, but are not limited to, Agaricus spp., Agaricus bisporus. Aspergillus spp., Aspergillus owamori, Aspergillus fumigatus, Aspergillus nidulans, Aspergillus niger, Aspergillus oryzae, Colletotrichum spp., Colletotrichum gloeosporiodes, Endothia spp., Endothia parasitica, Fusarium spp., Fusarium graminearum, Fusarium solani, Mucor spp., Mucor miehei, Mucor pusillus. Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii. Penicillium funiculo sum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus cells. In some cases, the host cell for expression of recombinant brazzein-53, recombinant brazzein-54, and/or recombinant monellin is a Pichia spp. cell, such as Pichia pastoris cell.

In some cases, a host cell selected for expression of a sweet polypeptide disclosed herein can be an insect cell. Suitable insect host cells include, but are not limited to, Sf9 cells from Spodoptera frugiperda, Sf21 cells from Spodoptera frugiperda, Hi-Five cells, BTI-TN-5B1-4 Trichophusiani cells, Schneider 2 (S2) cells from Drosophila melanogaster, and Schneider 3 (S3) cells from Drosophila melanogaster.

In some cases, a host cell selected for expression of a sweet polypeptide disclosed herein can be a mammalian cell. Non-limiting examples of mammalian host cells include HEK293 cells, HeLa cells, CHO cells, COS cells, Jurkat cells, NSO hybridoma cells, baby hamster kidney (BHK) cells, MDCK cells, NIH-3T3 fibroblast cells, and any other immortalized cell line derived from a mammalian cell.

In some cases, a sweet polypeptide described herein (e.g., brazzein-53, brazzein-54 and/or monellin (such as a single chain monellin)) can be produced in in vitro translation systems. An in vitro translation system generally refers to a translation system which is a cell-free extract containing elements for translation of an RNA molecule into a protein. An in vitro translation system can include ribosomes, tRNAs, initiator methionyl-tRNAMet, proteins or complexes involved in translation, e.g., eIF2, eIF3, the cap-binding (CB) complex, including the cap-binding protein (CBP) and eukaryotic initiation factor 4F (elF4F). A variety of in vitro translation systems are available. Non-limiting examples of in vitro translation systems include eukaryotic lysates, such as rabbit reticulocyte lysates, rabbit oocyte lysates, human cell lysates, insect cell lysates, and wheat gem extracts.

### Synthesis of Polypeptides

A sweet peptide described herein can be chemically synthesized. Methods of peptide chemical synthesis are known in the art. See, for example, Isidro-Llobet et. al. 2019. Journal of Organic Chemistry 84, 8, 4615-4628 or Stawikowski and Fields. 2002. Curr. Protoc. Protein Sci. doi: 10.1002/0471140864.ps1801s26.

### Recovery and Purification of Sweet Polypeptides

A sweet polypeptide such as recombinant brazzein-53, a recombinant brazzein-54, and/or a recombinant monellin described herein, can be expressed in a host cell, recovered, isolated, and/or purified from the supernatant of a cell culture, for example, if the expressed protein is secreted by the host cell into the cell culture medium. A sweet polypeptide described herein can, in some cases, be recovered from the host cell, for example, if the protein is expressed intracellularly. Proteins can be recovered from the intracellular space by disrupting the host cell, for example, by osmotic shock, sonication, or lysis. Once the cells are disrupted, cell debris can be removed by centrifugation or filtration, and the expressed proteins can be removed or recovered from the cell lysate.

In some cases, the sweet polypeptide can be produced using in vitro or cell-free protein synthesis systems, for example, using a cell-free translation system including a cell extract such as Escherichia coli cell extract, rabbit reticulocyte cell extract, wheat germ cell extract, or insect cell extract. The expressed protein can be recovered, isolated, and/or optionally purified from the cell extract by any suitable method.

A sweet polypeptide described herein can be recovered and/or isolated from supernatants and lysates by any of a variety of methods, including, but not limited to, chemical extraction, column chromatography, and filtration. The polypeptides can be purified using any of a variety of methods including liquid chromatography such as normal or reversed phase, high-performance liquid chromatography (HPLC), fast protein liquid chromatography (FPLC), and the like; affinity chromatography such as with inorganic ligands, monoclonal antibodies (e.g., immunoaffinity), and ion exchange (e.g., anion exchange, cation exchange); hydrophobic interaction chromatography: size exclusion chromatography; immobilized metal chelate chromatography; gel electrophoresis; ethanol precipitation; and any combination thereof. In some cases, the polypeptides can be purified by centrifugation and/or filtration, including sterile filtration, depth filtration, tangential flow filtration, ultrafiltration (UF), diafiltration (DF), and ultrafiltration/diafiltration (UF/DF).

A recovered and/or isolated sweet polypeptide described herein (e.g., brazzein-53) can be concentrated, dehydrated, dewatered, or dried by freeze drying, vacuum tray drying, spray drying, rotary drum drying, or any combination thereof. Drying can be done in a desiccator, vacuum dryer, conical dryer, spray dryer, fluid bed or any method known in the art. Preferably, methods are chosen that yield a dried product (e.g., a powder) with the greatest sweetness. The dried sweet protein can be optionally blended, pestled, milled, conched, pulverized, or other methods of processing known in the art. The resulting concentrated liquid or solid powder can be stored, diluted, rehydrated, or used in the formulation of food and beverage products, such as the tea compositions herein. In some cases, the concentrated liquid or solid powder can be stored at −20° C., 4° C., or room temperature without significant loss in sweetness. In some cases, the concentrated liquid can be diluted using water. In some cases, the solid powder can be rehydrated as a paste or a liquid using water or another liquid in which the solid powder is soluble.

Where the term “substantially purified” is used, this will refer to a composition in which the specific protein, peptide, or peptide forms the major component of the composition, such as constituting about 50% of the peptides in the composition or more. In preferred cases, a substantially purified peptide will constitute more than 60%, 70%, 80%, 90%, 95%, 99% or even more of the peptides in the composition.

A peptide, polypeptide or protein that is “purified to homogeneity.” as used herein, means that the peptide, polypeptide, or protein has a level of purity where the peptide, polypeptide or protein is substantially purified or free from other proteins/peptides and biological components. For example, a purified peptide, polypeptide, or protein will often be sufficiently free of other protein/peptide components so that degradative sequencing can be performed successfully.

Although preferred for use in some cases, there is no general requirement that the protein, polypeptide, or peptide always be provided in their most purified state. Indeed, it is contemplated that less substantially purified protein, polypeptide, or peptide, which are nonetheless enriched in the desired peptide compositions, relative to the natural state, will have utility in some cases. As used herein “enriched” refers to a component, such as a peptide or protein that is found at a high concentration in a solution or composition than in nature when produced the same way. Methods of enrichment of a component, such as enrichment of a peptide, are known in the art and are often similar to those of purification of components or peptides.

In other cases, a preparation enriched with any of the sweet peptides described herein can be used instead of a purified preparation. In this document, whenever purified is used, enriched can be used also. A preparation can be enriched not only by methods of purification, but also by the over-expression or over-production of the peptide by the host cell when compared to a wild-type host cell. This can be accomplished using recombinant methods, or by selecting conditions which will induce the expression of the peptide from the wild-type cells.

### Additives

The sweet proteins disclosed herein can be added to or mixed with one or more additives. In some cases, an additive can increase the shelf-life of the beverage product, improve the stability and consistency of sweetness and/or preserve flavor and taste over time.

### VI. Beverage Products Including a Sweet Protein

Beverage products described herein can be a beverage that includes brazzein-53, brazzein-54, monellin, and any combinations thereof. Non-limiting examples of a beverage product include a tea, or a tea and lemonade combination beverage (referred to as tea-lemonade). A tea can include a beverage comprising one or more of a black tea, a green tea, a matcha tea, a yellow tea, a purple tea, a pu-erh tea, a white tea, an oolong tea, an herbal tea, a fruit tea, or a combination thereof. In some cases, the tea can be a tea extract. In some cases, the tea can be a tea concentrate. A beverage product can include an iced tea, an unsweetened tea, an alcoholic tea, a hemp-infused tea, a prebiotic tea, a probiotic tea, or a bubble tea. In some cases, a tea includes some amount of fruit, fruit pulp, fruit juice, or fruit flavor. In some cases, a tea is a tea-lemonade and includes some amount of lemonade (e.g., between 10-90% lemonade, such as 40-60% lemonade or 50% lemonade-50% tea). Any of the beverage products described herein can further include an additional ingredient such as one or more of a plant-based sugar composition, an acid, a bulking agent, an emulsifier, and/or an additional flavoring agent. Any of the beverage products described herein, while further including an additional ingredient, specifically do not include a high intensity sweetener. In some cases, the beverage products, such as teas, described herein do not include sugar alternatives, stevia-derived sweetener, a robaudioside or an artificial sweetener such as aspartame or saccharin.

The beverage products herein can include additional flavoring agents. Additional flavoring agents can impart additional flavors or tastes. Non-limiting exemplary additional flavoring agents include fruit flavors (e.g., fruit juice, fruit puree, pieces of fruit, dried fruit, whole fruit, fruit flavor, fruit concentrate, fruit pulp, or an oil derived from a fruit), herbs such as mint, flavor-imparting oils, alcoholic beverages (e.g., vodka, malt liquor, whiskey, bourbon, or red wine), flavor extracts (e.g., toa flavor extracts). In some cases, a tea can be a flavored tea. In some cases, the flavored tea can be an herbal tea, which can include flavoring agents. Non-limiting flavor agents include anise seed, ashwagandha, barley, bergamot, butterfly pea, calendula, cardamom seed, chamomile, chicory, chrysanthemum, cinnamon, cloves, dandelion, Echinacea, elderberry flower, ginger, gingko, gotu kola, guava leaf, hibiscus, honeybush, iceland moss, jasmine, jiaogulan, kava, kuding, lapacho bark, lavender, lemon balm, lemongrass, licorice root, linden flower, marshmallow root, moringa, olive leaf, osmanthus, passionflower, peppermint, pine needle, raspberry leaf, rooibos, rose, rosehip, rosemary, sage, stinging nettle, thyme, tulsi, turmeric, valerian root, yarrow, yerba mate, or combinations thereof.

In some cases, a flavored tea can include a fruit flavored tea and/or tea-lemonade. A fruit flavored tea and/or tea-lemonade can include the flavor, the pulp, the oil, or the juice of any fruit as a flavoring agent. Non-limiting examples of flavoring agents that can impart a fruit flavor include banana, blood orange, cherries, citron, coconut, grape, grapefruit, key lime, kiwi, lemon, lime, mango, mulberries, nectarine, orange, orange peel, papaya, passionfruit, peach, pineapple, plums, pomegranate, raisins, raspberry, strawberry, yuzu, or combinations thereof. In some cases, a fruit flavored tea is a lemon tea beverage. In some cases, a fruit flavored tea is a mango yuzu tea beverage. In some cases, a fruit flavored tea is a peach tea beverage. In some cases, a fruit flavored tea is a raspberry tea beverage.

In some cases, any of the tea-lemonades described herein can include a flavored lemonade (e.g., a fruit-flavored lemonade). A tea-lemonade can include a lemon-flavored tea-lemonade, a lime-flavored tea-lemonade, other citrus tea-lemonade (e.g., yuzu, orange, citron, grapefruit), strawberry tea-lemonade, or raspberry-flavored tea-lemonade. In some cases, a tea-lemonade includes some amount of tea (e.g., between 10-90% tea, such as 40-60% tea).

Any of the flavoring agents described herein can be present in an amount between about 0.1% wt/wt and about 25% wt/wt (e.g., between about 0.4% wt/wt and about 0.6% wt/wt, or between about 3% wt/wt and about 4% wt/wt).

### Brazzein and Recombinant Sweet Proteins

The beverage products described herein, such as tea and tea-lemonade, contain sweet proteins, such as brazzein-53 and/or recombinant brazzein-53, brazzein-54 and/or recombinant brazzein-54, monellin and/or recombinant monellin, and combinations thereof as described herein.

In some cases, at least one sweet protein is added to any of the beverage products described herein as a powder, slurry, solution, or other preparation, and in some cases, the preparation contains other components in addition to the sweet protein. Within such preparations, the amount of sweet protein in the preparation can be from about 20% to about 99% wt/total preparation wt, also referred to herein as “purity” or “% pure”). For example, within such preparations, the amount of sweet protein in the preparation can be from about 50% purity to about 75% purity. In some cases, the preparation of sweet protein is at least 35% pure and/or no more than 85% pure. For example, the purity of sweet protein and/or recombinant sweet protein powder, before it is added to any of the beverage products described herein can be about 50%, about 55%, about 60%, about 65%, about 70%, or about 75% pure.

In some cases, any of the tea beverage products (e.g., teas or tea-lemonade combinations) described herein can include, from about 1 ppm to about 40 ppm of brazzein-53 and/or recombinant brazzein-53 protein in the beverage product. For example, the ppm of brazzein-53 protein and/or recombinant brazzein-53 protein can be about 1 ppm to about 40 ppm, about 1 ppm to about 39 ppm, about 1 ppm to about 38 ppm, about 1 ppm to about 37 ppm, about 1 ppm to about 36 ppm, about 1 ppm to about 35 ppm, about 1 ppm to about 34 ppm, about 1 ppm to about 33 ppm, about 1 ppm to about 32 ppm, about 1 ppm to about 31 ppm, about 1 ppm to about 30 ppm, about 1 ppm to about 29 ppm, about 1 ppm to about 28 ppm, about 1 ppm to about 27 ppm, about 1 ppm to about 26 ppm, about 1 ppm to about 25 ppm, about 1 ppm to about 24 ppm, about 1 ppm to about 23 ppm, about 1 ppm to about 22 ppm, about 1 ppm to about 21 ppm, about 1 ppm to about 20 ppm in the beverage product, such as a tea or tea-lemonade.

In some cases, the ppm of brazzein-53 protein and/or recombinant brazzein-53 protein can be about 5 ppm to about 40 ppm, about 5 ppm to about 39 ppm, about 5 ppm to about 38 ppm, about 5 ppm to about 37 ppm, about 5 ppm to about 36 ppm, about 5 ppm to about 35 ppm, about 5 ppm to about 34 ppm, about 5 ppm to about 33 ppm, about 5 ppm to about 32 ppm, about 5 ppm to about 31 ppm, about 5 ppm to about 30 ppm, about 5 ppm to about 29 ppm, about 5 ppm to about 28 ppm, about 5 ppm to about 27 ppm, about 5 ppm to about 26 ppm, about 5 ppm to about 25 ppm, about 5 ppm to about 24 ppm, about 5 ppm to about 23 ppm, about 5 ppm to about 22 ppm, about 5 ppm to about 21 ppm, about 5 ppm to about 20 ppm in the beverage product, such as a tea or tea-lemonade.

In some cases, the ppm of brazzein-53 protein and/or recombinant brazzein-53 protein can be about 8 ppm to about 62 ppm, about 8 ppm to about 61 ppm, about 8 ppm to about 60 ppm, about 8 ppm to about 59 ppm, about 8 ppm to about 58 ppm, about 8 ppm to about 57 ppm, about 8 ppm to about 56 ppm, about 8 ppm to about 55 ppm, about 8 ppm to about 54 ppm, about 8 ppm to about 53 ppm, about 8 ppm to about 52 ppm, about 8 ppm to about 51 ppm, about 8 ppm to about 50 ppm, about 8 ppm to about 49 ppm, about 8 ppm to about 48 ppm, about 8 ppm to about 47 ppm, about 8 ppm to about 46 ppm, about 8 ppm to about 45 ppm, about 8 ppm to about 44 ppm, about 8 ppm to about 43 ppm, about 8 ppm to about 42 ppm, about 8 ppm to about 41 ppm in the beverage product such as a tea or tea-lemonade.

In some cases, the ppm of brazzein-53 protein and/or recombinant brazzein-53 protein can be about 8 ppm to about 40 ppm, about 8 ppm to about 39 ppm, about 8 ppm to about 38 ppm, about 8 ppm to about 37 ppm, about 8 ppm to about 36 ppm, about 8 ppm to about 35 ppm, about 8 ppm to about 34 ppm, about 8 ppm to about 33 ppm, about 8 ppm to about 32 ppm, about 8 ppm to about 31 ppm, about 8 ppm to about 30 ppm, about 8 ppm to about 29 ppm, about 8 ppm to about 28 ppm, about 8 ppm to about 27 ppm, about 8 ppm to about 26 ppm, about 8 ppm to about 25 ppm, about 8 ppm to about 24 ppm, about 8 ppm to about 23 ppm, about 8 ppm to about 22 ppm, about 8 ppm to about 21 ppm, about 8 ppm to about 20 ppm in the beverage product such as a toa or tea-lemonade.

In some cases, the ppm of brazzein-53 protein and/or recombinant brazzein-53 protein can be about 10 ppm to about 65 ppm, about 10 ppm to about 60 ppm, about 10 ppm to about 50 ppm, about 10 ppm to about 45 ppm, about 10 ppm to about 40 ppm, about 10 ppm to about 39 ppm, about 10 ppm to about 38 ppm, about 10 ppm to about 37 ppm, about 10 ppm to about 36 ppm, about 10 ppm to about 35 ppm, about 10 ppm to about 34 ppm, about 10 ppm to about 33 ppm, about 10 ppm to about 32 ppm, about 10 ppm to about 31 ppm, about 10 ppm to about 30 ppm, about 10 ppm to about 29 ppm, about 10 ppm to about 28 ppm, about 10 ppm to about 27 ppm, about 10 ppm to about 26 ppm, about 10 ppm to about 25 ppm, about 10 ppm to about 24 ppm, about 10 ppm to about 23 ppm, about 10 ppm to about 22 ppm, about 10 ppm to about 21 ppm, about 10 ppm to about 20 ppm in the beverage product, such as a tea or tea-lemonade.

In some cases, the ppm of brazzein-53 protein and/or recombinant brazzein-53 protein can be about 10 ppm to about 30 ppm, about 10 to about 29, about 10 to about 28, about 10 to about 27, about 10 to about 26, about 10 to about 25, about 10 to about 24, about 10 to about 23, about 10 to about 22, about 10 to about 21, about 10 to about 20, about 10 to about 19, about 10 to about 18, about 10 to about 17, about 10 to about 16, about 10 to about 15, about 12 to about 30, about 12 to about 29, about 12 to about 28, about 12 to about 27, about 12 to about 26, about 12 to about 25, about 12 to about 24, about 12 to about 23, about 12 to about 22, about 12 to about 21, about 12 to about 20, about 12 to about 19, about 12 to about 18, about 12 to about 17, about 12 to about 16, about 12 to about 15, about 12 to about 27, or about 13 to about 26 in the beverage product, such as a tea or tea-lemonade.

In some cases, the brazzein-53 protein and/or recombinant brazzein-53 protein is the only sweet protein in a beverage and the amount of the brazzein-53 protein and/or recombinant brazzein-53 can be about 11 ppm, 12 ppm, 13 ppm, 14 ppm, 15 ppm, 16 ppm, 17 ppm, 18 ppm, 19 ppm, 20 ppm, 21 ppm, 22 ppm, 23 ppm, 26 ppm, 27 ppm, 28 ppm, 29 ppm, 30 ppm, 31 ppm, 32 ppm, 33 ppm 34 ppm, 35 ppm, 36 ppm, 43 ppm, 44 ppm, 45 ppm, 46 ppm, 47 ppm, 48 ppm, 49 ppm, 50 ppm, 51 ppm, 52 ppm, or 53 ppm in the beverage product such as a tea or tea-lemonade.

In some cases, the brazzein-53 protein and/or recombinant brazzein-53 protein is combined with one or more other sweet proteins (such as brazzein-54 or monellin) in a beverage and the amount of brazzein-53 protein and/or recombinant brazzein-53 protein can be about 1 ppm, 2 ppm, 3 ppm, 4 ppm, 5 ppm, 6 ppm, 7 ppm 8 ppm, 9 ppm, 10 ppm, 11 ppm, 12 ppm 13 ppm, 14 ppm, 15 ppm, 16 ppm, 17 ppm, 18 ppm, 19 ppm, 20 ppm, 21 ppm, 22 ppm, 23 ppm, 24 ppm, 25 ppm 26 ppm, 27 ppm, 28 ppm, or 29 ppm in the beverage product such as a tea or tea-lemonade.

In some embodiments, brazzein-53 protein and/or recombinant brazzein-53 protein is the only sweet protein in the beverage product. In some such cases, the beverage product is a tea and the amount of brazzein-53 protein and/or recombinant brazzein-53 protein can be about 8 ppm to about 62 ppm or about 16 ppm to about 31 ppm. In some such cases, the beverage product is a tea-lemonade and the amount of brazzein-53 protein and/or recombinant brazzein-53 protein can be about 24 ppm to about 96 ppm or about 48 ppm.

In some cases, brazzein-53 protein and/or recombinant brazzein-53 protein is combined with one or more other sweet proteins (such as brazzein-54 or monellin) in a beverage product. In some such cases, the beverage product is a tea and the amount of brazzein-53 protein and/or recombinant brazzein-53 protein can be about 1 ppm to about 27 ppm or about 5 ppm to about 16 ppm. In some such cases, the beverage product is a tea-lemonade and the amount of brazzein-53 protein and/or recombinant brazzein-53 protein can be about 4 ppm to about 42 ppm or about 16 ppm to about 24 ppm.

In some cases, the brazzein-54 protein and/or recombinant brazzein-54 protein is the only sweet protein in a beverage and the amount of the brazzein-54 protein and/or recombinant brazzein-54 can be about 19 ppm, 20 ppm, 21 ppm, 22 ppm, 23 ppm, 24 ppm, 25 ppm, 26 ppm, 27 ppm, 28 ppm, 29 ppm, 30 ppm, 31 ppm, 32, ppm, 41 ppm, 42 ppm, 43 ppm, 44 ppm, 45 ppm, 46 ppm, 47 ppm, 48 ppm, 49 ppm, 50 ppm, 51 ppm, 67 ppm, 68 ppm, 69 ppm, 70 ppm, 71 ppm, 72 ppm, 73 ppm, 74 ppm, 75 ppm, or 76 ppm in the beverage product such as a tea or tea-lemonade.

In some cases, the brazzein-54 protein and/or recombinant brazzein-54 protein is combined with one or more other sweet proteins (such as brazzein-53 or monellin) in a beverage and the amount of the brazzein-54 protein and/or recombinant brazzein-54 can be about 3 ppm, 4 ppm, 5 ppm, 6 ppm, 7 ppm, 8 ppm, 9 ppm, 10 ppm, 11 ppm, 12 ppm. 13 ppm 14 ppm, 15 ppm, 16 ppm, 17 ppm, 18 ppm, 19 ppm, 20 ppm, 21 ppm, 22 ppm, 23 ppm, 24 ppm, 25 ppm, 26 ppm, 27 ppm, 28 ppm, 29 ppm, 31 ppm, 32, ppm, 33 ppm, 34 ppm, 35 ppm, 36 ppm, 37 ppm, 38 ppm, 39 ppm, 40 ppm, 41 ppm, in the beverage product such as a tea or tea-lemonade.

In some embodiments, brazzein-54 protein and/or recombinant brazzein-54 protein is the only sweet protein in the beverage product. In some such cases, the beverage product is a tea and the amount of brazzein-54 protein and/or recombinant brazzein-54 protein can be about 12 ppm to about 92 ppm or about 24 ppm to about 46 ppm. In some such cases, the beverage product is a tea-lemonade and the amount of brazzein-54 protein and/or recombinant brazzein-54 protein can be about 35 ppm to about 142 ppm or about 71 ppm.

In some cases, brazzein-54 protein and/or recombinant brazzein-54 protein is combined with one or more other sweet proteins (such as brazzein-53 or monellin) in a beverage product. In some such cases, the beverage product is a tea and the amount of brazzein-54 protein and/or recombinant brazzein-54 protein can be about 2 ppm to about 40 ppm or about 8 ppm to about 23 ppm. In some such cases, the beverage product is a tea-lemonade and the amount of brazzein-54 protein and/or recombinant brazzein-54 protein can be about 6 ppm to about 62 ppm or about 23 ppm to about 36 ppm.

In some cases, the monellin protein and/or recombinant monellin protein is the only sweet protein in a beverage and the amount of the monellin protein and/or recombinant monellin can be about 25 ppm, 26 ppm, 27 ppm, 28 ppm, 29 ppm, 30 ppm. 31 ppm, 32 ppm, 33 ppm, 34 ppm, 35 ppm, 45 ppm, 46 ppm, 47 ppm 48 ppm, 49 ppm, 50 ppm, 51 ppm, 52 ppm, 53 ppm, 54 ppm, 55 ppm, 81 ppm, 82 ppm, 83 ppm, 84 ppm, 85 ppm, 86 ppm, 87 ppm, 88 ppm, 89 ppm, 90 ppm, or 91 ppm in the beverage product such as a tea or tea-lemonade.

In some cases, the monellin protein and/or recombinant monellin protein is combined with one or more other sweet proteins (such as brazzein-53 or brazzein-54) in a beverage and the amount of monellin protein and/or recombinant monellin protein can be about 5 ppm, 6 ppm, 7 ppm, 8 ppm, 9 ppm, 10 ppm, 11 ppm, 12 ppm, 13 ppm, 14 ppm, 15 ppm, 16 ppm, 17 ppm, 18 ppm, 19 ppm, 20 ppm, 21 ppm, 22 ppm, 23 ppm, 24 ppm, 25 ppm, 26 ppm, 27 ppm, 28 ppm, 29 ppm, 30 ppm, 31 ppm, 32 ppm, 33 ppm 34 ppm, 38 ppm, 39 ppm, 40 ppm, 41 ppm, 42 ppm, 43 ppm, 44 ppm, 45 ppm, 46 ppm, 47 ppm, or 48 ppm in the beverage product such as a tea or tea-lemonade.

In some embodiments, monellin protein and/or recombinant monellin protein is the only sweet protein in the beverage product. In some such cases, the beverage product is a tea and the amount of monellin protein and/or recombinant monellin protein can be about 15 ppm to about 100 ppm or about 29 ppm to about 50 ppm. In some such cases, the beverage product is a tea-lemonade and the amount of monellin protein and/or recombinant monellin protein can be about 43 ppm to about 173 ppm or about 86 ppm.

In some cases, monellin protein and/or recombinant monellin protein is combined with one or more other sweet proteins (such as brazzein-53 or brazzein-54) in a beverage product. In some such cases, the beverage product is a tea and the amount of monellin protein and/or recombinant monellin protein can be about 2 ppm to about 44 ppm or about 9 ppm to about 25 ppm. In some such cases, the beverage product is a tea-lemonade and the amount of monellin protein and/or recombinant monellin protein can be about 7 ppm to about 76 ppm or about 28 ppm to about 43 ppm.

In some cases, a beverage product includes brazzein-53 or recombinant brazzein-53 and monellin or recombinant monellin. In some cases, the ratio of brazzein-53 or recombinant brazzein-53 to monellin or recombinant monellin is from about 1:0.6 to about 1:5.4. In some cases, the ratio of brazzein-53 or recombinant brazzein-53 to monellin or recombinant monellin is from about 1:0.9 to about 1:3.1.

In some cases, a beverage product includes brazzein-54 or recombinant brazzein-54 and monellin or recombinant monellin. In some cases, the ratio of brazzein-54 or recombinant brazzein-54 to monellin or recombinant monellin is from about 1:0.4 to about 1:2.4. In some cases, the ratio of brazzein-54 or recombinant brazzein-54 to monellin or recombinant monellin is from about 1:0.6 to about 1:2.1.

In some cases, a beverage product includes brazzein-53 or recombinant brazzein-53 and brazzein-54 or recombinant brazzein-54. In some cases, the ratio of brazzein-53 or recombinant brazzein-53 to brazzein-54 or recombinant brazzein-54 is from about 1:0.2 to about 1:2.0. In some cases, the ratio of brazzein-53 or recombinant brazzein-53 to brazzein-54 or recombinant brazzein-54 is from about 1:0.3 to about 1:1.3.

In some cases, a beverage product includes brazzein-53 or recombinant brazzein-53, brazzein-54 or recombinant brazzein-54, and monellin or recombinant monellin. In some cases, the ratio of brazzein-53 or recombinant brazzein-53 to brazzein-54 or recombinant brazzein-54 to monellin or recombinant monellin is from about 1:0.2-2.1:0.6-5.4. In some cases, the ratio of brazzein-53 or recombinant brazzein-53 to brazzein-54 or recombinant brazzein-54 to monellin or recombinant monellin is from about 1:0.3-1.3:0.9-3.2.

### Sweeteners

In some cases, a sweetener can be added to the beverage product, such as a tea or tea-lemonade described herein. For example, a sweetener added to a beverage product described herein can include a plant-based sugar composition (also referred to herein as a plant-sugar). In some cases, the plant-based sugar composition is a low glycemic index sweetener. A low glycemic index sweetener can be added to the compositions described herein, for example, to improve organoleptic properties, such as a faster onset of sweetness compared to a composition without the low glycemic index sweetener. The glycemic index is a value assigned to foods based on how slowly or how quickly those foods cause increases in blood glucose level. Low glycemic index foods can achieve a more beneficial effect on blood glucose control, for example, in people with diabetes mellitus, and may also provide metabolic benefits for the general population. Methods for testing the glycemic index of a beverage have been described. See, for example, Wolever, et al. Nutrition Research 23:621-629, 2003. Low glycemic index foods, including low glycemic index sweeteners have a glycemic index of 55 or less.

Plant-based sugar compositions can be a low glycemic index sweetener and/or a natural sweetener. In some cases, the plant-based sugar composition includes only nature identical ingredients. Non-limiting exemplary plant-based sugar compositions include, agave, agave syrup. Jerusalem artichoke inulin, yacon, maple syrup, molasses, lucuma fruit, and monk fruit. In some cases, a plant-based sugar composition includes fructose, glucose, and combinations thereof. In some cases, the plant-based sugar compositions used herein for tea beverages and tea-lemonade beverages exclude stevia, erythritol, xylitol, rebaudiosides or any combinations thereof.

The plant-based sugar compositions can be present in any of the beverage products, such as the tea beverage and/or tea-lemonade beverages described herein. In some cases, the plant-based sugar composition is present in an amount between about 0.1% and about 10% volume/total volume, or any range included therein. For example, the plant-based sugar composition can be present in an amount between about 0.1% and about 5% vol/total vol, about 0.2% and about 5% vol/total vol, about 0.3% and about 5% vol/total vol, about 0.4% and about 5% vol/total vol, about 0.5% and about 5% vol/total vol, about 0.6% and about 5% vol/total vol, about 0.7% and about 5% vol/total vol, about 0.8% and about 5% vol/total vol, about 0.9% and about 5% vol/total vol, about 1% and about 5% vol/total vol, about 1% and about 4.9% vol/total vol, about 1% and about 4.8% vol/total vol, about 1% and about 4.7% vol/total vol, about 1% and about 4.6% vol/total vol, about 1% and about 4.6% vol/total vol, about 1% and about 4.5% vol/total vol, about 1% and about 4.4% vol/total vol, about 1% and about 4.3% vol/total vol, about 1% and about 4.2% vol/total vol, about 1% and about 4.1% vol/total vol, about 1% and about 4.0% vol/total vol, about 1% and about 3.9% vol/total vol, about 1% and about 3.8% vol/total vol, about 1% and about 3.7% vol/total vol, about 1% and about 3.6% vol/total vol, about 1% and about 3.5% vol/total vol, about 1% and about 3.4% vol/total vol, about 1% and about 3.3% vol/total vol, about 1% and about 3.2% vol/total vol, about 1% and about 3.1% vol/total vol, about 1% and about 3% vol/total vol.

In some cases, any of the beverage products described herein do not include sugar, such as cane sugar. In some cases, any of the beverage products described herein do not include any natural sweetener and/or any natural plant-based sugar composition. In some cases, the beverage products described herein do not include any high intensity sweeteners. Non-limiting examples of high intensity sweeteners include rebaudioside A, rebaudioside B, rebaudioside C, rebaudioside D, rebaudioside E, rebaudioside F, rebaudioside I; Rebaudioside J, rebaudioside K, rebaudioside M, rebaudioside N, rebaudioside O, rebaudioside Q, rebaudioside R, dulcoside A, dulcoside C; Rubusoside, steviol monoside, steviol bioside, stevioside, mogroside V, and saccharin. In some cases, the beverage products described herein, do not include allulose, aspartame, or sugar alcohols.

### Acid Components

In some cases, any of the beverage products, tea and tea-lemonade described herein can include preservative, an acid, and/or a pH adjuster. In some cases, the beverage product can include maleic acid, citric acid, lemon juice, and combinations thereof. Citric acid can be an additional flavoring agent, a preservative, an acid, a pH adjuster, any combination thereof. In some cases, any of the beverage products described herein have a pH between about 3 and about 5, for example, between about 3 and about 4, between about 3.1 and about 3.9, between about 3.2 and about 3.8, between about 3.3 and about 3.7, between about 3.3 and about 3.6, or about 3.0 and about 3.5

### BRIX

BRIX or degrees BRIX (° Bx), is a measure of dissolved solids in a liquid, and is sometimes used to measure dissolved sugar content of an aqueous solution. One degree BRIX is 1 gram of sucrose in 100 grams of solution. If the solution contains dissolved solids other than pure sucrose, then BRIX only approximates the dissolved solid content. BRIX can be used to quantify the strength of a solution as a percentage by mass. BRIX is typically determined by a refractometer or density meter. The beverage products described herein can have a BRIX less than soft drinks of juices containing sugar. In some cases, any of the beverage products described herein have a BRIX between about 1° Bx and about 6° Bx, between about 2° Bx and 6° Bx, between about 2° Bx and about 4° Bx, between about 1° Bx and about 2° Bx, between about 1° Bx and about 3° Bx, between about 2° Bx and about 3° Bx, or between about 2.3° Bx and about 3.7° Bx.

### Specific Gravity

Specific gravity, or relative density, is a measure of the density of a substance to the density of a reference material. For beverages, specific gravity is often the density of the substance in comparison to the density of water and is sometimes used to describe dissolved sugar content. As specific gravity is a relative measurement, specific gravity does not have units. Specific gravity can be determined by a hydrometer, pycnometer, or an oscillating U tube. In some cases, specific gravity can be calculated from a measurement of BRIX (see e.g., homebrewacademy.com/specific-gravity-to-brix/). In some cases, any of the beverage product described herein has a specific gravity between 1.000 and 1.020, or between about 1.020 and 1.015.

### V. Concentrates

In some aspects, beverage products described herein can be provided as a concentrate, i.e., in a concentrated form that can be diluted, such as with water, to provide a drinkable beverage product. In some cases, the concentrate is a 1× concentrate and is diluted 1:1 with water. In some cases, the concentrate is a 5× concentrate and is diluted 1:5 with water. In some cases, the concentrate is an 8× concentrate and is diluted 1:8 with water. The concentrate can be provided as a kit containing information describing directions for beverage product dilution, and optionally storage and consumption. Exemplary concentrates are provided herein. A concentrate can be a 1×, 2×, 3×, 4×, 5×, 6×, 7×, 8×, 9×, 10× concentrate. Such concentrates can be diluted with water at 1 part concentrate per 1, 2, 3, 4, 5, 6, 7, 8, 9, or 10 parts, respectively of water to create a drinkable (ready to consume) beverage product. In some cases, a concentrate can be more than 10×.

Exemplary tea and tea-lemonade concentrates are shown in Tables 6-13. In some cases, a concentrate includes a concentrated base formulation, such as shown in Tables 6, 8, 10 and 12 herein. In some cases, the sweet protein (e.g., brazzein-53, brazzein-54 and/or monellin) is added in concentrated amounts to the concentrated base formulation, such as shown in Tables 7, 9, 11 and 13 herein. Such concentrates when diluted with water then provide the drinkable tea or tea-lemonade having the desired amounts of the sweet proteins such as described herein.

### VI. Packaging

In some aspects, beverage products described herein can be packaged into a container. In some cases, beverage products described herein can be packaged in a container selected from the group consisting of: a box, a tube, a jar, a vial, a bag, a pouch, a drum, a bottle, and a can. The container can contain information describing directions for beverage product consumption.

### Definitions

The term “promoter” is a nucleic acid sequence that is operably linked to a nucleic acid sequence encoding a polypeptide (e.g., a recombinant peptide) that can increase the transcription of the nucleic acid sequence encoding the polypeptide (e.g., a peptide described herein). In some cases, a promoter is constitutive. In other cases, a promoter is inducible. Non-limiting examples of promoters are described herein. Additional examples of promoters are known in the art.

The term “culturing” refers to growing a population of cells, e.g., microbial cells, under suitable conditions for growth, in a liquid or solid medium.

The term “purifying” means a step performed to isolate a recombinant peptide from one or more other impurities (e.g., bulk impurities) or components present in a fluid containing a recombinant peptide (e.g., liquid culture medium polypeptides or one or more other components (e.g., DNA, RNA, other polypeptides, endotoxins, viruses, etc.) present in or secreted from a mammalian cell).

The terms “isolated”, “purified”, “separated”, and “recovered” as used herein refer to a material (e.g., a polypeptide, nucleic acid, or cell) that is removed from at least one component with which it is naturally associated, for example, at a concentration of at least 90% by weight, or at least 95% by weight, or at least 98% by weight of the sample in which it is contained. For example, these terms can refer to a material which is substantially or essentially free from components which normally accompany it as found in its native state, such as, for example, an intact biological system, or is substantially or essentially free from other proteins in the system from which it is expressed.

As used herein, the term “host cell” refers to a cell or cell line into which a recombinant expression vector (e.g., a nucleic acid construct) can be introduced for expression of the peptide in the host cell. A host cell including a recombinant vector can be referred to as a “recombinant host cell.”

The term “ppm”, as used herein, means parts-per-million and is a weight relative parameter. A part-per-million is a microgram per gram, such that a component that is present at 10 ppm is present at 10 micrograms of the specific component per 1 gram of the aggregate mixture.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 to and including 22.

Unless otherwise required by context, singular terms shall include pluralities and plural terms shall include the singular. As used herein, the singular form “a”, “an”, and “the” include plural references unless indicated otherwise. For example, “an” excipient includes one or more excipients.

### EXAMPLES

Various aspects of the disclosure are further illustrated by the following non-limiting examples.

### Examples 1-4

To assess the effects of sweet protein, recombinant brazzein-53 was formulated in tea beverages and tea-lemonade beverages. For these examples, the recombinant brazzein-53 was expressed in Pichia, and purified to about 65% purity and stored as a powder that was then used as an ingredient in the formulations exemplified in Examples 1-4.

### Example 1. Sweet Protein Formulation for a Lemon Black Tea Beverage

Ingredients were pre-weighed ingredients as set forth in Table 2. The brazzein-53 preparation and citric acid were added and mixed until the brazzein-53 and citric acid were dissolved. The agave syrup, black tea extracts and lemon juice concentrate were added and the combined ingredients were mixed until homogeneous. The flavors were then mixed in until homogeneous. The pH and brix were then measured. The mixture was kept under constant agitation to prevent sedimentation. The completed tea mixture was filled into containers (e.g., 16 oz. cans), nitrogen dosed, and lids applied. The containers were tunnel pasteurized to an internal temperature of 160° F. for 10 minutes and then cooled to <100° F. before storage.

TABLE 2
Lemon Black Tea. Unless otherwise stated, values are in weight %.

| Water | 92.73 | |
|---|---|---|
| Black Tea Extract | 2.15 | |
| Brewed Black Tea Extract | 2.15 | |
| Agave Syrup | 2.14 | |
| Sweetness Enhancer Flavor | 0.50 | |
| Meyer Lemon | 0.10 | |
| Black Tea Flavoring | 0.10 | |
| Lemon Juice Concentrate | 0.08 | |
| Citric Acid | 0.05 | |
| Brazzein-53 protein | 10-16 | ppm |
| pH | 3.2-3.5 | |
| T.A. (as citric) | 0.13% | |
| BRIX | 2.6-2.8 | |
| Specific Gravity (g/ml) | 1.01 | |
| Density (lbs/gallon) | 8.43 | |
| Total Sugar/16 fl oz. | 7.44 | g |
| Sugar from Agave | 7.28 | g |

In some cases, teas were made with 13 ppm or 14 ppm of brazzein-53 protein. In some cases, the BRIX measured was between 2.6 and 2.8. In some cases, the BRIX was 2.8, 2.9, or 3.0. In some cases, the pH was between 3.2-3.5. In some cases, the pH was 3.25 or 3.46. In some cases, the pH was 3.28, 3.35, 3.36, 3.37, or 3.4.

### Example 2. Sweet Protein Formulation for a Tea-Lemonade (Half Lemonade Half Tea) Beverage

Ingredients were pre-weighed ingredients as set forth in Table 3. The brazzein-53 preparation was added and mixed until the brazzein-53 was dissolved. The agave Syrup, Black Tea Extract, and Single Strength Lemon Juice were added and the combination was mixed until homogeneous. The flavors were then added and further mixed until homogeneous. The pH and brix were measured. To avoid sedimentation, the mixture was kept under constant agitation. The completed tea-lemonade mixture was filled into containers (e.g., 16 oz. cans), nitrogen dosed, and lids applied. The containers were tunnel pasteurized to an internal temperature of 160° F. for 10 minutes and then cooled to <100° F. before storage.

TABLE 3
Half Lemonade and Half Tea. Unless otherwise
stated, values are in weight %.

| Water | 92.41 | |
|---|---|---|
| Black Tea Extract | 2.65 | |
| Lemon Juice-Single Strength | 2.40 | |
| Agave Syrup | 2.05 | |
| Sweetness Enhancer Flavor | 0.50 | |
| Brazzein-53 protein | 22-30 | ppm |
| pH | 3.3-3.4 | |
| T.A. (as citric acid) | 0.21% | |
| BRIX | 2.6-3.6 | |
| Specific Gravity (g/ml) | 1.01 | |
| Density (lbs/gallon) | 8.43 | |
| Total Sugar/16 fl oz. | 7.36 | g |
| Sugar from Agave | 6.97 | g |

In some cases, tea-lemonade was made with about 25 ppm of brazzein-53 protein.

### Example 3. Sweet Protein Formulation for a Mango Yuzu Green Tea Beverage

Ingredients were pre-weighed ingredients as set forth in Table 4. The brazzein-53 preparation and citric acid were added and mixed until the brazzein-53 and citric acid were dissolved. The agave syrup, green tea extract, mango puree, and yuzu juice were added and the combined ingredients were mixed until the mixture was homogeneous. The flavors and green tea essence were then mixed in until homogeneous. The pH and brix were then measured. The mixture was kept under constant agitation to prevent sedimentation. The completed tea mixture was filled into containers (e.g., 16 oz. cans), nitrogen dosed, and lids applied. The containers were tunnel pasteurized to an internal temperature of 160° F. for 10 minutes and then cooled to <100° F. before storage. In some cases, teas were made with 13 ppm or 14 ppm of brazzein-53 protein.

TABLE 4
Mango Yuzu Green Tea. Unless otherwise
stated, values are in weight %.

| Water | 92.43 | |
|---|---|---|
| Mango Puree Single Strength | 2.50 | |
| Organic Green Tea Leaf Extract | 1.50 | |
| Agave Syrup | 1.50 | |
| Yuzu Juice NFC, Single Strength | 0.66 | |
| Sweetness Enhancer Flavor | 0.50 | |
| Mango Flavor Nat Type Organic | 0.50 | |
| Pure Green Tea Essence | 0.35 | |
| Citric Acid | 0.06 | |
| Brazzein-53 protein | 10-16 | ppm |
| pH | 3.3-3.4 | |
| T.A. (as citric acid) | 0.16% | |
| BRIX | 2.1-3.0 | |
| Specific Gravity (g/ml) | 1.01 | |
| Density (lbs/gallon) | 8.40 | |
| Total Sugar/16 fl oz. | 7.06 | g |
| Sugar from Agave | 5.09 | g |

In some cases, teas were made with about 13 ppm or about 14 ppm of brazzein-53 protein. In some cases, the BRIX was 2.1 or 2.3. In some cases, the BRIX was 2.8 or 3.0. In some cases, the pH was 3.46 or 3.34. In some cases, the pH was 3.2, 3.34, 3.37, 3.4, or 3.49.

### Example 4. Sweet Protein Formulation for a Peach Black Tea Beverage

Ingredients were pre-weighed ingredients as set forth in Table 5. The brazzein-53 preparation and citric acid were added and mixed until the brazzein-53, malic acid, and citric acid were dissolved. The agave syrup, black tea extract, peach juice concentrate, and peach puree concentrate were added and the combined ingredients were mixed until the batch was homogeneous. The flavors were then mixed in until homogeneous. The pH and brix were then measured. The mixture was kept under constant agitation to prevent sedimentation. The completed tea mixture was filled into containers (e.g., 16 oz. cans), nitrogen dosed, and lids applied. The containers were tunnel pasteurized to an internal temperature of 160° F. for 10 minutes and then cooled to <100° F. before storage.

TABLE 5
Peach Black Tea. Unless otherwise stated, values are in weight %.

| Water | 93.41 | |
|---|---|---|
| Black Tea Extract | 2.5 | |
| Agave Syrup | 1.15 | |
| Peach Juice Concentrate | 1.0 | |
| Peach Freestone Puree | 1.0 | |
| 32° Concentrate | | |
| Sweetness Enhancer Flavor | 0.51 | |
| Black Tea Flavoring | 0.20 | |
| Natural Peach Flavor | 0.16 | |
| Malic Acid | 0.06 | |
| Citric Acid | 0.02 | |
| Brazzein-53 protein | 22-30 | ppm |
| pH | 3.4-3.6 | |
| T.A. (as citric acid) | 0.23% | |
| BRIX | 2.9-3.7 | |
| Specific Gravity (g/ml) | 1.01 | |
| Density (lbs/gallon) | 8.42 | |
| Total Sugar/16 fl oz. | 7.27 | g |
| Sugar from Agave | 3.91 | g |

In some cases, teas were made with 25 ppm or 26 ppm of brazzein-53 protein. In some cases, BRIX was 3.53 or 3.5. In some cases, the BRIX was 3.5, 3.6, 3.7 or 3.8. In some cases, the pH was 3.36. In some cases, the pH was 3.43, 3.45, 3.47, 3.51, 3.52, or 3.55.

### Examples 5-8

For Examples 5-8, formulations using recombinant brazzein-53, recombinant brazzein 54 and recombinant monellin alone and in combination were designed to achieve a sweetness benchmark of tea beverages and tea-lemonade beverages. For these examples, the recombinant sweet proteins were each expressed in Pichia, and purified to about 40-55% purity and stored as a powder that was then used as an ingredient in the formulations exemplified in Examples 5-8. The amounts of recombinant sweet protein used in the Examples are expressed as ppm of pure protein.

### Example 5. Lemon Black Tea and Concentrates

Lemon black tea base was formulated as a drinkable beverage and in 3 concentrated formulations. The concentrated formulations can be diluted with water as follows to create the drinkable beverages: 1×=1:1 concentrate:water, 5×: 1:5 concentrate:water, 8×1:8 concentrate:water. Unless otherwise stated, the ingredients are listed by % weight to total weight.

TABLE 6
Lemon Black Tea Formulations.

| | | CONCENTRATES | | |
|---|---|---|---|---|
| | | (% = weight of | | |
| BASE FORMULATION | | ingredient per total weight) | | |
| Ingredients | Tea | 1X | 5X | 8X |
| Water | 93.07% | 86.13% | 58.39% | 37.59% |
| Natural Black Tea Extract | 3.30% | 6.60% | 19.80% | 29.70% |
| Agave Syrup | 2.45% | 4.90% | 14.70% | 22.05% |
| Brewed Black Tea Extract | 0.25% | 0.49% | 1.48% | 2.21% |
| Natural Type Sweetness | 0.57% | 1.14% | 3.42% | 5.13% |
| Enhancer Flavor | | | | |
| Natural Lemon Meyer | 0.11% | 0.22% | 0.66% | 0.99% |
| WONF | | | | |
| TasteEssentials ™ Nat | 0.11% | 0.22% | 0.66% | 0.99% |
| Black Tea Flavor Type | | | | |
| Lemon Juice Concentrate, | 0.09% | 0.18% | 0.55% | 0.83% |
| 400 GPL | | | | |
| Citric Acid | 0.06% | 0.11% | 0.34% | 0.51% |

The drinkable beverage was created with brazzein-53 and benchmarked with a formulation of the brazzein-53 containing lemon black tea with brazzein-53 at 16 ppm. Amounts of brazzein-53 for the concentrate formulations were calculated based on the drinkable beverage formulation. The amounts of brazzein-54 and monellin used in the formulations (shown in Table 7) were calculated based on the sweetness profile of brazzein-54 and monellin as compared to brazzein-53 in the formulation. A small panel of 3-5 tasters was used to confirm that sweetness was similar to the benchmark.

The comparison of the single proteins in the tea indicated different sweetness profiles. Brazzein-53 provided good sweetness, with a rounded character. The upper range gave significantly more linger as compared to the lower range, but the increase in sweetness was not as pronounced. Brazzein-54 provided a rounded character at a level that provided similar sweetness to the benchmark and tasters favored it in sweetness. Monellin had a faster sweetness onset as compared to the other sweet proteins, but very little linger in this tea formulation. The combination of all 3 proteins provided a rounded character with very similar sweetness compared to the benchmark.

TABLE 7
Sweet protein ranges (in ppm) for lemon black tea and concentrates

| | BRZ53 | BRZ54 | MON |
|---|---|---|---|
| Tea | | | |
| Single Proteins | 8-32 | | — |
| | — | 12-48 | — |
| | — | — | 15-58 |
| 2-proteins | 2-14 | 3-21 | — |
| | 2-14 | — | 4-25 |
| | — | 3-21 | 4-25 |
| 3-proteins | 1-9 | 2-14 | 2-17 |
| 1X Concentrate | | | |
| Single Proteins | 16-64 | | — |
| | — | 24-95 | — |
| | — | — | 29-116 |
| 2-proteins | 4-28 | 6-41 | — |
| | 4-28 | — | 7-51 |
| | — | 6-41 | 7-51 |
| 3-proteins | 3-18 | 4-27 | 5-33 |
| 5X Concentrate | | | |
| Single Proteins | 47-188 | | — |
| | — | 70-278 | — |
| | — | — | 85-341 |
| 2-proteins | 12-82 | 17-122 | — |
| | 12-82 | — | 21-149 |
| | — | 17-122 | 21-149 |
| 3-proteins | 8-54 | 11-80 | 14-99 |
| 8X Concentrate | | | |
| Single Proteins | 73-291 | | — |
| | — | 107-430 | — |
| | — | — | 132-527 |
| 2-proteins | 18-127 | 27-188 | — |
| | 18-127 | — | 33-231 |
| | — | 27-188 | 33-231 |
| 3-proteins | 12-84 | 18-124 | 22-152 |

### Example 6. Mango Yuzu Tea and Concentrates

Mango yuzu tea base was formulated as a drinkable beverage and in 3 concentrated formulations as shown in Table 8. The concentrated formulations can be diluted with water as follows to create the drinkable beverages: 1×=1:1 concentrate:water, 5×: 1:5 concentrate:water, 8×1:8 concentrate:water. Unless otherwise stated, the ingredients are listed by % weight to total weight.

TABLE 8
Mango Yuzu Tea Formulations

| BASE FORMULATION | | CONCENTRATES | | |
|---|---|---|---|---|
| MANGO YUZU BASE | | 1X | 5X | 8X |
| Water | 90.14% | 80.30% | 40.89% | 11.33% |
| Mango Puree, Single | 3.25% | 6.51% | 19.52% | 29.28% |
| Strength | | | | |
| Organic Green Tea Leaf | 1.95% | 3.90% | 11.71% | 17.57% |
| Extract | | | | |
| Agave Syrup | 1.95% | 3.90% | 11.71% | 17.57% |
| Yuzu Juice NFC, Single | 0.86% | 1.72% | 5.15% | 7.73% |
| Strength | | | | |
| Natural Type Sweetness | 0.65% | 1.30% | 3.90% | 5.86% |
| Enhancer Flavor | | | | |
| Mango Flavor Nat Type | 0.65% | 1.30% | 3.90% | 5.86% |
| Organic Certified | | | | |
| Pure Green Tea Essence | 0.46% | 0.91% | 2.73% | 4.10% |
| Citric Acid | 0.078% | 0.16% | 0.47% | 0.70% |

The drinkable beverage was created with brazzein-53 and benchmarked with a formulation of the brazzein-53 containing mango yuzu tea with brazzein-53 at 18 ppm. Amounts of brazzein-53 for the concentrate formulations were calculated based on the drinkable beverage formulation. The amounts of brazzein-54 and monellin used in the formulations (shown in Table 9) were calculated based on the sweetness profile of brazzein-54 and monellin as compared to brazzein-53 in the formulation. A small panel of 3-5 tasters was used to confirm that sweetness was similar to the benchmark.

The single proteins teas were compared for sweetness characteristics. Similar to the lemon black tea, the single brazzein-53 protein provided good sweetness with a rounded character. The upper range had more linger than the lower range. The single brazzein-54 also provided a sweetness with a rounded character that was similar to the benchmark. Monellin gave some delay in the sweetness as well as some linger. The combination of all 3 sweet proteins gave an upfront sweetness, that was flavorful with a slight linger.

TABLE 9
Sweet protein ranges (in ppm) for mango yuzu tea and concentrates

| | BRZ53 | BRZ54 | MON |
|---|---|---|---|
| Original Teas | | | |
| Single Proteins | 9-36 | | — |
| | — | 14-54 | — |
| | — | — | 15-60 |
| 2-proteins | 2-16 | 3-24 | — |
| | 2-16 | — | 4-26 |
| | — | 3-24 | 4-26 |
| 3-proteins | 1-10 | 2-16 | 2-17 |
| 1X CONCENTRATE | | | |
| Single Proteins | 18-72 | | — |
| | — | 27-108 | — |
| | — | — | 30-120 |
| 2-proteins | 5-32 | 7-47 | — |
| | 5-32 | — | 8-53 |
| | — | 7-47 | 8-53 |
| 3-proteins | 3-21 | 4-31 | 5-35 |
| 5X CONCENTRATE | | | |
| Single Proteins | 53-212 | | — |
| | — | 79-318 | — |
| | — | — | 88-353 |
| 2-proteins | 13-93 | 20-139 | — |
| | 13-93 | — | 22-154 |
| | — | 20-139 | 22-154 |
| 3-proteins | 9-61 | 13-92 | 15-102 |
| 8X CONCENTRATE | | | |
| Single Proteins | 82-327 | | — |
| | — | 123-491 | — |
| | — | — | 136-545 |
| 2-proteins | 20-143 | 31-215 | — |
| | 20-143 | — | 34-239 |
| | — | 31-215 | 34-239 |
| 3-proteins | 14-95 | 20-142 | 23-159 |

### Example 7. Fruit-Flavored Black Tea and Concentrates

Peach black tea base was formulated as a drinkable beverage and in 3 concentrated formulations as shown in Table 10A. The concentrated formulations can be diluted with water as follows to create the drinkable beverages: 1×=1:1 concentrate:water, 5×: 1:5 concentrate:water, 8×1:8 concentrate:water. Unless otherwise stated, the ingredients are listed by % weight to total weight.

TABLE 10A
Peach black tea and concentrate formulations

| BASE FORMULATION | | CONCENTRATES | | |
|---|---|---|---|---|
| PEACH BLACK TEA BASE | | 1X | 5X | 8X |
| Water | 92.08% | 84.22% | 52.53% | 28.79% |
| Natural Black Tea Extract | 3.00% | 6.00% | 18.01% | 27.01% |
| Agave Syrup | 1.38% | 2.76% | 8.28% | 12.43% |
| Peach Juice Concentrate | 1.20% | 2.40% | 7.20% | 10.81% |
| Peach Freestone Puree 32° | 1.20% | 2.40% | 7.20% | 10.81% |
| Concentrate. Aseptic | | | | |
| Natural Type Sweetness | 0.61% | 1.22% | 3.67% | 5.51% |
| Enhancer Flavor | | | | |
| TasteEssentials ™ Nat | 0.24% | 0.48% | 1.44% | 2.16% |
| Black Tea Flavor Type | | | | |
| Natural Peach Flavor | 0.19% | 0.37% | 1.12% | 1.68% |
| WONF #29897 | | | | |
| L-Malic Acid, Organic | 0.067% | 0.13% | 0.40% | 0.60% |
| Compliant & NGPV | | | | |
| Citric Acid | 0.023% | 0.046% | 0.14% | 0.21% |

Other fruit-flavored toas and concentrates were created based on the peach black tea formulation. For example, a raspberry black tea and raspberry black tea concentrates were formulated based on the respective peach black tea formulations, substituting red raspberry juice concentrate for the peach concentrate, red raspberry puree concentrate for the peach puree concentrate, and natural raspberry flavor (WONF) for the natural peach flavor (WONF), and omitting the acid components. The raspberry black tea base was formulated as a drinkable beverage and in 3 concentrated formulations as shown in Table 10B. The concentrated formulations can be diluted with water as follows to create the drinkable beverages: 1×=1:1 concentrate:water, 5×: 1:5 concentrate:water, 8×1:8 concentrate:water. Unless otherwise stated, the ingredients are listed by % weight to total weight.

TABLE 10B
Raspberry black tea and concentrate formulations

| BASE FORMULATION | | CONCENTRATES | | |
|---|---|---|---|---|
| Ingredients | Tea | 1X | 5X | 8X |
| Water | 92.23% | 84.48% | 53.44% | 30.16% |
| Natural Black Tea Extract | 3.20% | 6.40% | 19.20% | 28.80% |
| Agave Syrup | 1.57% | 3.14% | 9.42% | 14.13% |
| Red Raspberry Juice | 1.20% | 2.40% | 7.20% | 10.80% |
| Concentrate 65 Brix | | | | |
| Red Raspberry Puree | 0.75% | 1.50% | 4.50% | 6.75% |
| Concentrate 28 Brix | | | | |
| Natural Type Sweetness | 0.61% | 1.22% | 3.66% | 5.49% |
| Enhancer Flavor | | | | |
| TasteEssentials ™ Nat | 0.24% | 0.48% | 1.44% | 2.16% |
| Black Tea Flavor Type | | | | |
| Raspberry Flavor Natural | 0.19% | 0.38% | 1.14% | 1.71% |
| WONF | | | | |

The drinkable beverages were created with brazzein-53 and benchmarked with a formulation of the brazzein-53 containing peach black tea with brazzein-53 at 32 ppm and raspberry back tea with brazzein-53 at 31 ppm. Amounts of brazzein-53 for the concentrate formulations were calculated based on the drinkable beverage formulation. The amounts of brazzein-54 and monellin used in the formulations (shown in Table 11) were calculated based on the sweetness profile of brazzein-54 and monellin as compared to brazzein-53 in the formulation. A small panel of 3-5 tasters was used to confirm that sweetness was similar to the benchmark.

The single protein teas were compared for sweetness characteristics. The single brazzein-53 produced a tart tea with a good sweetness level and rounded character. Like the other tea formulations, the upper range of protein gave more linger than the lower range. The single brazzein-54 tea produced a rounded sweetness character similar to the benchmark. Monellin alone gave a similar sweetness to the tea made with the brazzeins but had a bit of linger. The combination of all 3 proteins produced a tea that was less tart, rounded and in general good flavor. However, the single brazzein 53 was preferred by the tasters as compared to the combination in this formulation.

TABLE 11
Sweet protein ranges (in ppm) for fruit-flavored black
tea drinkable beverage and diluted concentrates

| | BRZ53 | BRZ54 | MON |
|---|---|---|---|
| Original Teas | | | |
| Single Proteins | 16-62 | | — |
| | — | 23-92 | — |
| | — | — | 25-100 |
| 2-proteins | 4-27 | 6-40 | — |
| | 4-27 | — | 6-44 |
| | — | 6-40 | 6-44 |
| 3-proteins | 3-18 | 4-26 | 4-29 |
| 1X Concentrate | | | |
| Single Proteins | 31-124 | | — |
| | — | 46-183 | — |
| | — | — | 50-200 |
| 2-proteins | 8-54 | 11-80 | — |
| | 8-54 | — | 13-88 |
| | — | 11-80 | 13-88 |
| 3-proteins | 5-36 | 8-53 | 8-58 |
| 5X Concentrate | | | |
| Single Proteins | 91-365 | | — |
| | — | 135-539 | — |
| | — | — | 147-588 |
| 2-proteins | 23-160 | 34-236 | — |
| | 23-160 | — | 37-257 |
| | — | 34-236 | 37-257 |
| 3-proteins | 15-105 | 22-156 | 24-170 |
| 8X Concentrate | | | |
| Single Proteins | 141-564 | | — |
| | — | 208-833 | — |
| | — | — | 227-909 |
| 2-proteins | 35-247 | 52-364 | — |
| | 35-247 | — | 57-398 |
| | — | 52-364 | 57-398 |
| 3-proteins | 23-163 | 34-240 | 38-263 |

### Example 8. Tea-Lemonade and Concentrates

A tea-lemonade base was formulated as a drinkable beverage and in 3 concentrated formulations as shown in Table 12. The concentrated formulations can be diluted with water as follows to create the drinkable beverages: 1×=1:1 concentrate:water, 5×: 1:5 concentrate:water, 8×1:8 concentrate:water. Unless otherwise stated, the ingredients are listed by % weight to total weight.

TABLE 12
Tea-Lemonade Formulations

| BASE FORMULATION | | CONCENTRATES | | |
|---|---|---|---|---|
| Tea-Lemonade BASE | | 1X | 5X | 8X |
| Water | 92.91% | 85.81% | 57.44% | 36.16% |
| Natural Black Tea Extract | 2.83% | 5.66% | 16.98% | 25.47% |
| Agave Syrup | 2.64% | 5.27% | 15.81% | 23.72% |
| Natural Type Sweetness | 0.64% | 1.29% | 3.86% | 5.78% |
| Enhancer Flavor | | | | |
| Lemon Puree | 0.50% | 0.99% | 2.97% | 4.46% |
| Lemon Pulp Cells | 0.39% | 0.78% | 2.34% | 3.51% |
| ALL NAT Lemon FL WONF | 0.10% | 0.20% | 0.60% | 0.90% |
| Lemonade Type 37328 | | | | |

The drinkable beverage was created with brazzein-53 and benchmarked with a formulation of the containing tea-lemonade with brazzein-53 at 48 ppm. Amounts of brazzein-53 for the concentrate formulations were calculated based on the drinkable beverage formulation. The amounts of brazzein-54 and monellin used in the formulations (shown in Table 13) were calculated based on the sweetness profile of brazzein-54 and monellin as compared to brazzein-53 in the formulation. A small panel of 3-5 tasters was used to confirm that sweetness was similar to the benchmark.

TABLE 13
Sweet protein ranges (in ppm) for tea-lemonade and concentrates

| | BRZ53 | BRZ54 | MON |
|---|---|---|---|
| Tea-Lemonade | | | |
| Single Proteins | 24-96 | | — |
| | — | 35-142 | — |
| | — | — | 43-173 |
| 2-proteins | 6-42 | 9-62 | — |
| | 6-42 | — | 11-76 |
| | — | 9-62 | 11-76 |
| 3-proteins | 4-28 | 6-41 | 7-50 |
| 1X Concentrate | | | |
| Single Proteins | 48-192 | | — |
| | — | 71-284 | — |
| | — | — | 86-346 |
| 2-proteins | 12-84 | 18-124 | — |
| | 12-84 | — | 22-151 |
| | — | 18-124 | 22-151 |
| 3-proteins | 8-55 | 12-82 | 14-100 |
| 5X Concentrate | | | |
| Single Proteins | 141-565 | | — |
| | — | 209-834 | — |
| | — | — | 254-1016 |
| 2-proteins | 35-247 | 52-365 | — |
| | 35-247 | — | 64-445 |
| | — | 52-365 | 64-445 |
| 3-proteins | 23-163 | 34-241 | 42-294 |
| 8X Concentrate | | | |
| Single Proteins | 218-873 | | — |
| | — | 322-1289 | — |
| | — | — | 393-1571 |
| 2-proteins | 55-382 | 81-564 | — |
| | 55-382 | — | 98-687 |
| | — | 81-564 | 98-687 |
| 3-proteins | 36-252 | 53-372 | 65-454 |

What is claimed is:

1. A tea beverage comprising a tea, a plant-based sugar composition and a recombinant brazzein, wherein the recombinant brazzein is present in an amount between 8 ppm and 40 ppm, and wherein the plant-based sugar composition is present in an amount between 1-3% (vol/total vol).

2. The tea beverage of claim 1, wherein the recombinant brazzein is present in an amount between 10 ppm and 30 ppm.

3. The tea beverage of claim 1 or claim 2, wherein the recombinant brazzein is recombinant brazzein-53.

4. The tea beverage of any one of claims 1-3, wherein the recombinant brazzein is produced by a yeast or filamentous fungus.

5. The tea beverage of any one of claims 1-3, wherein the plant-based sugar composition comprises agave syrup.

6. The tea beverage of any one of claims 1-4, wherein the plant-based sugar composition comprises fructose and glucose.

7. The tea beverage of any one of claims 1-5, wherein the tea beverage does not include any high intensity sweeteners selected from the group consisting of rebaudioside A, rebaudioside B, rebaudioside C, rebaudioside D, rebaudioside E, rebaudioside F, rebaudioside I; Rebaudioside J, rebaudioside K, rebaudioside M, rebaudioside N, rebaudioside O, rebaudioside Q, rebaudioside R, dulcoside A, dulcoside C; Rubusoside, steviol monoside, steviol bioside, stevioside, mogroside V, allulose, aspartame, sugar alcohols, and saccharin.

8. The tea beverage of any one of claims 1-7, further comprising an acid component.

9. The tea beverage of claim 8, wherein the acid component is selected from malic acid, citric acid, and lemon juice.

10. The tea beverage of any one of claims 1-9, farther comprising a flavoring agent.

11. The tea beverage of any one of claims 1-10, further comprising a fruit puree.

12. The tea beverage of any one of claims 1-10, wherein the tea is a black tea, a green tea, a white tea, or any combination thereof.

13. The tea beverage of any one of claims 1-12, wherein the BRIX is between 2° Bx and 3° Bx.

14. The tea beverage of any one of claims 1-12, wherein the pH is between 3 and 5, between 3 and 4, or between 3.3 and 3.6.

15. The tea beverage of any one of claims 1-12, wherein the specific gravity is between 1.000 and 1.020 or between about 1.010 and 1.015.

16. The tea beverage of any one of claims 1-15, wherein the composition does not comprise cane sugar.

17. A tea beverage comprising a tea, a plant-based sugar composition and at least one recombinant sweet protein, wherein the recombinant sweet protein is present in an amount between 1 ppm and 175 ppm, and wherein the plant-based sugar composition is present in an amount between 1-3% (vol/total vol).

18. The tea beverage of claim 17, wherein the at least one recombinant sweet protein comprises brazzein-53.

19. The tea beverage of claim 18, wherein the brazzein-53 is present in an amount between 1 ppm and 96 ppm.

20. The tea beverage of claim 18, wherein the brazzein-53 is present in an amount between 5 ppm and 48 ppm.

21. The tea beverage of claim 20, wherein the brazzein-53 is the only sweet protein in the tea beverage.

22. The tea beverage of claim 21, wherein the brazzein-53 is present in an amount between 8 ppm and 96 ppm or between 16 ppm and 48 ppm.

23. The tea beverage of any one of claims 18-20, wherein the tea beverage further comprises recombinant brazzein-54.

24. The tea beverage of claim 22, wherein the brazzein-54 is present in an amount between 2 ppm and 62 ppm.

25. The tea beverage of claim 24, wherein the brazzein-54 is present in an amount between 8 ppm and 36 ppm.

26. The tea beverage of any one of claim 18-20 or 23-25, wherein the tea beverage further comprises recombinant monellin.

27. The tea beverage of claim 26, wherein the monellin is present in an amount between 2 ppm and 76 ppm.

28. The tea beverage of claim 26, wherein the monellin is present in an amount between 9 ppm and 43 ppm.

29. The tea beverage of claim 17, wherein the at least one recombinant sweet protein comprises brazzein-54.

30. The tea beverage of claim 29, wherein the brazzein-54 is present in an amount between 2 ppm and 142 ppm.

31. The tea beverage of claim 29, wherein the brazzein-54 is present in an amount between 8 ppm and 71 ppm.

32. The tea beverage of claim 29, wherein the brazzein-54 is the only sweet protein in the tea beverage.

33. The tea beverage of claim 32, wherein the brazzein-54 is present in an amount between 12 ppm and 142 ppm or between 24 ppm and 71 ppm.

34. The tea beverage of any one of claims 29-31, wherein the tea beverage further comprises recombinant monellin.

35. The tea beverage of claim 34, wherein the monellin is present in an amount between 2 ppm and 76 ppm.

36. The tea beverage of claim 34, wherein the monellin is present in an amount between 9 ppm and 43 ppm.

37. The tea beverage of claim 17, wherein the at least one recombinant sweet protein comprises monellin.

38. The tea beverage of claim 37, wherein the monellin is present is an amount between 2 ppm and 173 ppm.

39. The tea beverage of claim 37, wherein the monellin is present is an amount between 9 ppm and 86 ppm.

40. The tea beverage of claim 37, wherein the monellin is the only sweet protein in the tea beverage.

41. The tea beverage of claim 37, wherein the monellin is present is an amount between 15 ppm and 173 ppm.

42. The tea beverage of claim 37, wherein the monellin is present is an amount between 29 ppm and 86 ppm.

43. The tea beverage of any one of claims 17-42, wherein the tea is a black tea, a green tea, a white tea, a fruit-flavored tea or any combination thereof.

44. The tea beverage of claim 17, wherein the tea beverage is a tea-lemonade and wherein:
a. the at least one sweet protein is recombinant brazzein-53 in an amount between 4 ppm and 96 ppm; or
b. the at least one sweet protein is recombinant brazzein-54 in an amount between 6 ppm and 142 ppm, or
c. the at least one sweet protein is recombinant monellin in an amount between 7 ppm and 173 ppm.

45. The tea beverage of claim 17, wherein the tea beverage is a tea-lemonade, wherein the at least one sweet protein comprises a single sweet protein and wherein:
a. the single sweet protein is recombinant brazzein-53 in an amount between 24 ppm and 96 ppm; or
b. the single sweet protein is recombinant brazzein-54 in an amount between 35 ppm and 142 ppm; or
c. the single sweet protein is recombinant monellin in an amount between 43 ppm and 173 ppm.

46. The tea beverage of any one of claims 17-45, further comprising a flavoring.

47. The tea beverage of any one of claims 17-45, further comprising a fruit puree.

48. The tea beverage of any one of claims 17-47, wherein the at least one recombinant sweet protein is produced by a microbial organism.

49. The tea beverage of claim 48, wherein the microbial organism is a yeast or a filamentous fungus.

50. The tea beverage of claim 48 or claim 49, wherein the microbial organism is a Pichia sp.

51. The tea beverage of any one of claims 17-50, wherein the plant-based sugar composition comprises agave syrup.

52. The tea beverage of any one of claims 17-50, wherein the plant-based sugar composition comprises fructose and glucose.

53. The tea beverage of any one of claims 17-52, wherein the tea beverage does not include any high intensity sweeteners selected from the group consisting of rebaudioside A, rebaudioside B, rebaudioside C, rebaudioside D, rebaudioside E, rebaudioside F, rebaudioside I; Rebaudioside J, rebaudioside K, rebaudioside M, rebaudioside N, rebaudioside O, rebaudioside Q, rebaudioside R, dulcoside A, dulcoside C; Rubusoside, steviol monoside, steviol bioside, stevioside, mogroside V, saccharin, allulose, aspartame, and sugar alcohols.

54. The tea beverage of any one of claims 17-53, further comprising an acid component.

55. The tea beverage of claim 54, wherein the acid component is selected from malic acid, citric acid, and lemon juice.

56. The tea beverage of any one of claims 17-43 or claims 46-55, wherein the tea is a black tea, a green tea, a white tea, or any combination thereof.

57. The tea beverage of any one of claims 17-56, wherein the BRIX is between 2° Bx and 4.3° Bx, between 2° Bx and 3° Bx, or between 3.9° Bx and 4.3° Bx.

58. The tea beverage of any one of claims 17-57, wherein the pH is between 3 and 5, between 3 and 4, or between 3.3 and 3.6.

59. The tea beverage of any one of claims 17-58, wherein the specific gravity is between 1.000 and 1.020 or between about 1.010 and 1.015.

60. The tea beverage of any one of claims 17-59, wherein the composition does not comprise cane sugar.

61. A tea concentrate, wherein when diluted with water results in a tea beverage as set forth in any one of claims 1-60.

62. The tea concentrate of claim 60, wherein the concentrate is between 1× and 10× concentrated as compared to the tea beverage.


SEQUENCE LISTING (5 sequences)

SEQ ID NO: 1 - protein, 53 aa, Pentadiplandra brazzeana
DKCKKVYENYPVSKCQLANQCNYDCKLDKHARSGECFYDEKRNLQCICDYCEY

SEQ ID NO: 2 - protein, 54 aa, Pentadiplandra brazzeana
XDKCKKVYENYPVSKCQLANQCNYDCKLDKHARSGECFYDEKRNLQCICDYCEY

SEQ ID NO: 3 - protein, 45 aa, Dioscoreophyllum cumminsii
FREIKGYEYQLYVYASDKLFRADISEDYKTRGRKLLRFNGPVPPP

SEQ ID NO: 4 - protein, 50 aa, Dioscoreophyllum cumminsii
GEWEIIDIGPFTQNLGKFAVDEENKIGQYGRLTFNKVIRPCMKKTIYEEN

SEQ ID NO: 5 - protein, 96 aa, Dioscoreophyllum cumminsii
GEWEIIDIGPFTQNLGKFAVDEENKIGQYGRLTFNKVIRPCMKKTIYEENGFREIKGYEYQLYVYASDKLFRADISEDYKTRGRKLLRFNGPVPPP